(19) United States
(12) Patent Application Publication
(10) Pub. No.: US 2009/0263870 A1
(43) Pub. Date: Oct. 22, 2009

(54) SYSTEM AND METHOD FOR AMPLIFYING A NUCLEIC ACID MOLECULE

(75) Inventors: Juergen Pipper, Singapore (SG); Pavel Neuzil, Singapore (SG); Yi Zhang, Chendu (CN); Muhammad Khidhir Khairudin, Singapore (SG); Tseng-Ming Hsieh, Singapore (SG)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: AGENCY FOR SCIENCE, TECHNOLOGY AND RESEARCH, Singapore (SG)

(21) Appl. No.: 12/428,901

(22) Filed: Apr. 23, 2009

Related U.S. Application Data
(63) Continuation of application No. 12/208,079, filed on Sep. 10, 2008.
(60) Provisional application No. 60/935,968, filed on Sep. 10, 2007.
(60) Provisional application No. 60/960,871, filed on Oct. 17, 2007.
(60) Provisional application No. 61/136,284, filed on Aug. 25, 2008.

(51) Int. Cl. C12P 19/34; C12M 1/00

(52) U.S. Cl. 435/91.2; 435/289.1

(57) ABSTRACT

There is provided a method and/or system which allow on-chip preconditioning of complex real-world samples and/or handling of limited amounts of target material, and/or on-chip nucleic acid amplification process, using a free droplet containing magnetic attractable material. The nucleic acid amplification process comprises controlling the position of the magnetic attractable material and performing the nucleic acid amplification in a thermocycling droplet located onto at least one temperature zone. The low thermal masses of the herein described heaters/temperature sensors come along with fast temperature transitions within the corresponding temperature zones allowing impressing temperature gradients in at least one temperature zone between subsequent or within the same thermocycle(s). Additionally, the variable residence times of the droplet in a given temperature zone permit to customize the denaturation, annealing and/or extension times within the same or between different PCR runs. Additionally, the herein described method and/or system allow amplification of one or more nucleic acid molecules. Additionally, the herein described method and/or system allow real-time monitoring with or without the presence of magnetic attractable material bound to said nucleic acid molecule.

### RELATED APPLICATIONS

This application is a continuation of U.S. patent application Ser. No. 12/208,079, filed Sep. 10, 2008 and claims the benefit of U.S. Provisional application No. 60/935,968, filed Sep. 10, 2007, of U.S. Provisional application No. 60/960,871, filed Oct. 17, 2007, and of U.S. Provisional application 61/136,284, filed Aug. 25, 2008, the contents of which are incorporated herein in their entirety by reference.

### FIELD OF THE INVENTION

This application relates to a system or method for amplifying a nucleic acid molecule. More particularly, it relates to on-chip preconditioning of complex real-world samples and/or handling of limited amounts of target material, and/or on-chip nucleic acid molecule amplification process.

### BACKGROUND OF THE INVENTION

The following review is merely provided to aid in the understanding of the present invention and neither it nor any of the references cited within it are admitted to be prior art to the present invention.

Miniaturization of devices in the chemical, pharmaceutical and biotechnological field has lead to the development of microfluidic devices that control the flow of liquid and permit the performance of a number of chemical and biological reactions.

Without purification and/or preconcentration, biological samples, such as whole blood, urine, saliva or faeces cannot be efficiently analyzed in a microfluidic environment. Solids, particulates, air bubbles, erythrocytes, RNases, DNases, salts, etc. in most cases have to be removed, because they generally tend to interfere with the microfluidic operations, downstream applications and/or subsequent analysis. These processes are highly dependent on the nature of the sample and are not necessarily small in scale.

Lehmann et al. (Angew. Chem. Int., Ed. 2006, 45, 3062-3067) relates to an integrated on-chip DNA purification process which uses off-chip pre-purified and pre-lysed material as a DNA source. Droplets containing magnetic particles and the pre-purified and pre-lysed material are immersed in a modified silicone oil-filled reservoir to perform bio(chemical) processes, and are submitted to a combination of inhomogenous electromagnetic field (coils) together with a homogenous magnetic field (external permanent magnet). However, Lehmann et al. do not enable the ability to separate/purify/isolate starting material and/or reaction products from crude or complex mixtures, Lehmann et al. also do not use free droplets.

Lee et al. (Lab Chip, 2006, 6, 886-895) relates to a method and device for DNA extraction from isolated cells and subsequent real-time detection in a single microchip by combining laser irradiation, magnetic beads and real-time polymerase chain reaction (PCR) within a microchamber on a single microchip. PCR is conducted in a real-time PCR machine using the same microchip, after laser irradiation in a hand-held device equipped with a small laser diode. The magnetic beads are used to bind proteins and contaminants and remove these from the sampling solution by the use of a magnet. However, Lee et al. does not enable the ability to separate/purify/isolate starting material and/or reaction products from crude or complex mixtures followed by a further processing within the same machine.

Several lab-on-a-chip (LOC), micro total analysis system (TAS), and biological microelectromechanical systems (BioMEMS) have been developed for moving, merging/mixing, splitting, and heating of droplets on surfaces, such as electrowetting-on-dielectric (EWOD) [Pollack, M. G. et al., Appl. Phys. Lett. (2000), 77, 1725-1726], surface acoustic waves (SAW) [Wixforth, A. et al., mstnews (2002), 5, 42-43], dielectrophoresis [Cascoyne, P. R. C. et al., Lab-on-a-Chip (2004), 4, 299-309], and locally asymmetric environments [Daniel, S. et al., Langmuir (2005), 21, 4240-4228].

However, these methods lack the most important operation for performing sequential biological processes: the ability to separate/purify/isolate starting material and/or reaction products from crude or complex mixtures. In order to permit such a separation a solid phase needs to be introduced as part of the droplet-based system.

The design of an interface that allows, for instance, the on-chip preconditioning of complex real-world samples and/or the handling of limited amounts of target material on a single chip still remains elusive. It would be advantageous to provide such interface, for instance, for monitoring potential outbreak, (e.g. (re)emerging infectious or parasitic diseases like influenza, HIV/AIDS, cholera, malaria, tuberculosis or measles) in some of the developing Asian, Middle Eastern, and African countries, where adequate instrumentation and/or diagnostic test kits, for instance, for sample collection, isolation, RT-PCR, and gel electrophoresis are either unaffordable or restricted to a few laboratories.

Furthermore, diagnostic methods to detect outbreak-causing target material often require reliable and fast PCR for DNA or reverse transcription PCR (RT-PCR) for RNA detection. Often, real-time PCR also known as quantitative PCR (qPCR) is preferred. Most bench-scale thermocyclers depend on a thermoelectrically heated metal block holding plastic tubes with up to 50 μL PCR mixture. This set-up results in a high thermal mass, and the run time is dominated by the temperature transition rates between single thermocycling steps. Typically, 45 thermocycles of a 300-base pair (bp) amplicon take more than one hour. Downscaling and taking advantage of materials with a high heat conductivity address this issue—a (sub)microscale on-chip PCR is completed within minutes owing to its fast heat and mass transfer (Neuzil et al., Nucleic Acid Research, 2006, 34: 11, e77).

There are two ways for conducting an on-chip PCR (Roper et al., Anal. Chem., 2005, 77, 3887-3894). In a first way, in the “time domain”, the PCR mixture is kept stationary and the chip chamber is typically cycled between three different temperatures. This format is a direct miniaturization of a flexible bench-scale thermocycler using resistive, inductive, convective, or infrared (IR) heat sources. The “time domain” PCR allows for customizing temperatures of annealing, denaturation or extension steps, for instance incrementally in- or decreasing the temperatures during these steps. Such customizing enables running touch down PCR (Don, et al., 1991, Nucleic Acids Res., 19 (14): 4008), prolonging the denaturation of genomic DNA in early thermocycles, activating different types of hot start DNA polymerase, etc.

In a second way, in the “space domain”, a PCR mixture is driven through different zones on the chip, which zones are constantly held at three different temperatures. The PCR mixture is typically driven by pneumatical, thermosiphonal, electrokinetical or magnetical means through unidirectional, bidirectional, spiral or circular inflexible microcapillary or microchannel. Such process allows fast thermal equilibrium of the PCR mixture and thus, PCR in the space domain allows for fast thermocycling.

Fast temperature changes within an individual temperature zone have been ignored in past “space domain” PCR strategies, which have all relied on an inflexible microchannel-inspired chip design (Nakano et al., 1994, Biosci. Biotechnol. Biochem., 58, 349-352; Kopp et al., 1998, Science, 280, 1046-1048; Obeid et al., 2003, Anal. Chem., 75, 288-295; Chabert et al., 2006, 78, 7722-7728; Chiou, 2001, Anal. Chem., 73, 2018-2021; Hashimoto et al., 2004, Lab Chip, 4, 638-645; Liu et al., 2002, Electrophoresis, 23, 1531-1536; Wang et al., 2005, J. Micromech. Microeng., 15, 1369-1377; Wang, et al., 2007, J. Micromech. Microeng., 17, 367-375; Chen et al., 2004, Anal. Chem., 76, 3707-3715; Chen et al., 2005, Anal. Chem., 77, 658-666; Sun et al., 2007, Lab Chip, 10.1039/b700575j.).

It would therefore be advantageous to have a method and/or system which would allow on-chip preconditioning of complex real-world samples and/or handling of limited amounts of target material, and/or on-chip nucleic acid amplification process. It would also be advantageous, in the context of on-chip nucleic acid amplification process, that such method and/or system would include the above advantageous features of the “time domain” and/or of the “space domain” PCR.

### SUMMARY OF THE INVENTION

Accordingly, there is provided in a broad aspect, a method and/or system which allows on-chip preconditioning of complex real-world samples and/or handling of limited amounts of target material, and/or on-chip nucleic acid amplification.

In one aspect, the present invention provides a method for amplifying a nucleic acid molecule, said method comprising: providing a fluid droplet, said fluid droplet comprising an inner phase and an outer phase, wherein the outer phase is immiscible with the inner phase, and the outer phase is surrounding the inner phase, the inner phase comprises sample comprising or suspected of comprising said nucleic acid molecule, the inner phase is shielded from the environment by the outer phase, and said inner phase comprises surface functionalized magnetically attractable matter; providing at least one surface; providing at least a heater for heating a respective temperature zone on said at least one surface; disposing said fluid droplet onto said at least one surface; and processing said nucleic acid molecule on said at least one surface, said processing comprising (i) controlling the position of said magnetically attractable matter relative to said at least one surface so as to purify said nucleic acid molecule; and (ii) amplifying said nucleic acid molecule, said amplifying comprising locating said magnetically attractable matter onto said temperature zone.

In one aspect, the present invention provides a system for amplifying a nucleic acid molecule, said system comprising: at least one surface for receiving a first fluid droplet, said fluid droplet comprising an inner phase and an outer phase, wherein the outer phase is immiscible with the inner phase, and the outer phase is surrounding the inner phase, wherein the inner phase comprises a sample comprising or suspected of comprising said nucleic acid molecule, and the inner phase is shielded from the environment by the outer phase, wherein said inner phase comprises surface functionalized magnetically attractable matter, at least one heater for heating a respective temperature zone on said at least one surface; means for controlling the position of said magnetically attractable matter relative to said surface so as to (1) purify said nucleic acid molecule; and (2) locate said magnetically attractable matter onto said temperature zone; and means for amplifying said nucleic acid molecule.

In one aspect, the present invention provides A method for amplifying a nucleic acid molecule, said method comprising (a) providing at least one surface for receiving a sample comprising or suspected of comprising said nucleic acid molecule, said at least one surface comprising a plurality of temperature zones at which temperature can be independently regulated, each temperature zone being located at a different location on said at least one surface; (b) disposing a sample onto said at least one surface; and (c) amplifying said nucleic acid molecule by moving said sample between said plurality of temperature zones, wherein said sample has a residency time at each temperature zone which is independently controlled.

In one embodiment, the herein described on-chip nucleic acid amplification includes the above advantageous features of either or both the “time domain” and the “space domain” PCR.

In one embodiment, the whole preconditioning and/or nucleic acid amplification process is performed on a single disposable chip.

In one embodiment, the above defined preconditioning of complex real-world samples comprises dilution, mixing, isolation, concentration, purification, and the like, of target material and/or nucleic acid molecule.

In one embodiment, the above defined subsequent nucleic acid amplification process comprises any one of a reverse-transcription (RT), polymerase chain reaction (PCR), RT-PCR, real-time PCR also called quantitative (qPCR), real-time RT-PCR (qRT-PCR), isothermal amplification methods, such as helicase dependent amplidication (tHDA), smart amplification process (SMAP), loop-mediated amplification (LAMP), rolling circle amplification (RCA), recombinase polymerase amplification (RPA), and the like.

In one embodiment, the heaters used in accordance with the invention comprise Platinum or silicon as described respectively in WO 2007/094739 and Neuzil et al. (supra).

The following examples are presented in order to provide a more detailed description of specific embodiments of the represent invention and are not to be construed as limiting the scope of the invention.

### BRIEF DESCRIPTION OF THE DRAWINGS

In the accompanying drawings, which illustrate, by way of example only, embodiments of the present invention, in which:

FIG. 1 Shows block diagram of an embodiment of a PCR chip of the invention placed on the same printed circuit board (PCB). In this embodiment, up to four heaters are independently controlled.

FIG. 2 Shows a calibration of an embodiment of a PCR chip of the invention. The voltage outputs of every temperature sensor for three separate measurements were plotted versus the temperature. The lines are linear regression fits to the voltage outputs of the four temperature sensors with y=B*x+A (A and B are correlation coefficients). During the calibration, the top PCB accommodating the PCR chip and reference TSic-706-temperature sensor (Innovative Sensor Technologies) was immersed into FC-70 Fluorinert (3M) at different temperatures between 40-100° C.

FIG. 3 Shows a GFP-transfected THP-1 cells immunocaptured onto Dynabeads CD15. Most of the cells were buried inside the bulk of the superparamagnetic particles' slurry. Scale bar, 10 μm.

FIG. 4 Shows a sample preparation and qPCR according to an embodiment of the invention. (a) After immunocapture via their CD15 cell surface marker, (b, c) the Dynabeads CD15-bound GFP-transfected THP-1 cells were extracted/preconcentrated from the blood droplet and (d-l) purified two times in washing solution droplets. (d-h) Potential PCR inhibitors eventually enclosed in the Dynabeads CD15 slurry, such as erythrocytes, remained in the washing solution. The sequence a-l is functional equivalent to a macroscopic filter or centrifuge, in which the stationary and mobile phases are inverted. (m) Thereafter, the purified GFP-transfected THP-1 cells were merged with the qPCR mixture droplet and thermally lysed in the temperature zone 1. (n) Finally, the qPCR took place in the space domain by rotating/pausing the droplet over different temperature zones (heated locations). Interrogating the droplet every turn in the extension zone by a detector enabled real-time monitoring. G Teflon-coated glass substrate; 2 blood droplet spiked with GFP-transfected THP-1 cells; 3 and 4 washing solution droplets; 1 superparamagnetic particles with immunocaptured GFP-transfected THP-1 cells; H₁-H₄ heaters/temperature (T)-sensors; D fluorescence detector. The arrows indicate the start/end point of the sample preparation and qPCR, respectively. Scale bar, 5 mm.

FIG. 5 Shows a trajectory for the first thermocycle according to an embodiment of the invention. After completing the cell lysis in the temperature zone 1 (corresponding to heater/T-sensor 1: H₁), the droplet was first linearly moved to the temperature zone 3 (corresponding to heater/T-sensor 3: H₃), and then clockwise rotated back to H₁ via temperature zone 4 (corresponding to heater/T-sensor 4: H₄). This approach enables a simple microfluidic actuation by coupling a circular and linear movement. Otherwise, a rotation has to be superpositioned with a two-dimensional movement to realize of a full rotation for the very first thermocycle. (a) For thermocycling protocol 1, the heater(s) 1, 2, as well as 3 and 4 were at 95, 60, and 72° C., respectively. Note that the thermocycling profile is distorted. (b) For thermocycling protocol 2, the heater(s) 1 and 2-4 were at 95 and 60° C., respectively. Since the linear movement takes 3 s, the thermocycling profile is barely distorted. (c) For thermocycling protocol 3, the heaters 1 and 3 as well as 2 and 4 were at 95 and 60° C., respectively. 1 Dynabeads CD15; 2 blood droplet spiked with GFP-transfected THP-1 cells; 3 and 4 washing solution droplets; H₁-H₄ heaters/T-sensors; D fluorescence detector.

FIG. 6 Shows a schematic of a set-up according to an embodiment of the invention. (PCB 3) printed circuit board 3; (R) reference temperature sensor for calibrating the PCR chip; (H₁-H₄) heaters/T-sensors 1-4; (G) Teflon-coated glass substrate; (D) rotating droplet containing a template DNA and PCR mixture; (M) permanent magnet glued to a (Di) rotating disc; (S) stepper motor fixed to a (Ma) macroscopic x, y-stage for coupling a circular and linear movement; (0) long distance objective of a fluorescence microscope coupled with a photomultiplier tube. Real-time monitoring was accomplished while the droplet paused for 1-4 s in the focus of the objective in the extension zone located on top of the temperature zone 4 (corresponding to heater/T-sensor 4: H₄). Other PCBs contain the microelectronics for the thermal management and the optics using a miniaturized fluorescence detection system as well as the power supply.

FIG. 7 Shows a droplet formation and actuation according to an embodiment of the invention. The superparamagnetic particles are suspended in an aqueous solution and encapsulated in an immiscible liquid on a Teflon-coated glass substrate. A permanent magnet moderates moving, merging, mixing, and splitting of the droplets. (G) Teflon-coated glass substrate; (B) aqueous buffer; (Su) superparamagnetic particles; (I) immiscible liquid; (M) permanent magnet.

FIG. 8 Shows a droplet arrangement according to an embodiment of the invention. (PCB 3) printed circuit board 3; (H₁-H₄) heaters/T-sensors 1-4; (G) Teflon-coated glass substrate; (M) permanent magnet fixed to a stepper motor; (1) 250 mL droplet containing Dynabeads CD15; (2) 25 μL blood droplet spiked with around 30 GFP-transfected THP-1 cells; (3) and (4) 25 μL droplets comprising washing solutions; (5) 1.5 μL droplet holding the PCR mixture emulsified in 4.5 μL mineral oil. Not shown is the fluorescence detector positioned above the extension zone corresponding to the temperature zone 4 (corresponding to heater/T-sensor 4: H₄). Scale bar, 10 mm.

FIG. 9 Shows a steady-state temperature distribution on a Teflon-coated glass substrate for the thermocycling protocol 2 using ANSYS 10 according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 95.0, 60.0, 60.0, and 60.0° C. for the temperature zones 1-4 (corresponding to heater/T-sensor 1-4: H₁-H₄), respectively.

FIG. 10 Shows an IR-image of the temperature distribution on a Teflon-coated glass substrate for the thermocycling protocol 2 according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 94.5, 60.6, 60.4, and 60.6° C. for the temperature zones 1-4 (corresponding to heater/T-sensor 1-4: H₁-H₄), respectively. Scale bar, 10 mm.

FIG. 11 Shows a temperature transition between the temperature zones 1 and 2 (corresponding to heater/T-sensor 1 and 2: H₁-H₂) along the droplet track extracted from FIG. 10 and FIGS. 14 and 16 according to an embodiment of the invention. (a) The dashed/dotted (-•-•) line shows the thermocycling protocol 1. (b) The solid line shows the thermocycling protocol 2. (c) The dotted line (------) represents the thermocycling protocol 3. A rotation of 90° translated into a traveling distance of 5.8 mm.

FIG. 12 Shows a RT-PCR according to an embodiment of the invention. The solid line is the NTC. (b) The solid line shows the thermocycling protocol 1. (c) The dashed (----) line represents the thermocycling protocol 2. A negative control was also performed (-•-•). The C_{Ts} were 28.2 and 30.4, respectively.

FIG. 13 Shows a steady-state temperature distribution on a Teflon-coated glass substrate for the thermocycling protocol 1 using ANSYS 10 according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 95.0, 60.0, 72.0, and 72.0° C. for the temperature zones 1-4 (corresponding to heater/T-sensor 1-4: H₁-H₄), respectively.

FIG. 14 Shows an IR-image of a Teflon-coated glass substrate according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 94.6, 61.1, 72.4, and 72.1° C. for the temperature zones 1-4 (corresponding to heater/T-sensor 1-4: H₁-H₄), respectively. Scale bar, 10 mm.

FIG. 15 Shows a steady-state temperature distribution on a Teflon-coated glass substrate for the thermocycling protocol 3 using ANSYS 10 according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 95.0, 60.0, 95.0, and 60.0° C. for the temperature zones 1-4 (corresponding to heater/T-sensor 1-4: H₁-H₄), respectively.

FIG. 16 Shows an IR-image of a Teflon-coated glass substrate according to an embodiment of the invention. The four heaters/T-sensors, traveling path of the droplet, and droplet containing the Dynabeads CD15/PCR mixture () emulsified in mineral oil ( ) are superpositioned. The mean temperatures within the inner rings are 94.4, 60.5, 95.1, and 60.8° C. for the temperature zones 1-4 (corresponding to heater/T-sensor 1-4: H₁-H₄), respectively. Scale bar, 10 mm.

FIG. 17 Shows a capillary electrophoresis of PCR products according to an embodiment of the invention. (M1) marker 15 bp; (M2) marker 600 bp; (D) primer dimer; (P) PCR product, 99 bp. The dashed line (----) is the negative control. (a) The dashed/dotted line (-•-•) shows the three-step thermocycling protocol using SYBR Green I. (b) The dashed line represents the two-step thermocycling protocol 1. (c) The solid line represents the two-step thermocycling protocol 2. Typically, the yield of the PCR products was about 20-40 ng μL⁻¹. For all these experiments around 30 GFP-transfected THP-1 cells were used as input.

FIG. 18 Shows a manipulation of droplets by magnetic forces according to an embodiment of the invention. (a) The droplet self-assembles on a perfluorinated surface by overlaying an aqueous suspension of superparamagnetic particles with mineral oil. A permanent magnet is used to (b) move, (c) merge, (d) mix, and (e-g) split droplets. Every droplet and/or droplet manipulation represents an item, equipment or task in a biological laboratory. For example, the sequence—merge, mix, and split—emulates a two-dimensional solid phase extraction (SPE). The dead volume is negligible and buffers are easily exchangeable. Silica-coated superparamagnetic particles enable the specific isolation, purification, and preconcentration of nucleic acids. (h) After SPE, the immobilized viral RNA is magnetically pulled out of the raw sample solution, washed four times for removing residual contaminants, and desorbed into a RT-PCR solution positioned on top of a miniaturized real-time thermocycler (see also FIG. 19). The mineral oil prevents the aqueous phase from evaporating at temperatures of up to 150° C. (G) perfluorinated glass substrate; (M) permanent magnet; (Su) superparamagnetic particles; (B) aqueous buffer; (I) thin film of immiscible liquid; (N) nucleocapsid containing HPAI H5N1 RNA; (L) lysis buffer; (Sa) raw sample solution; (WI) and (W2) washing solutions; (R) RT-PCR mixture; (T) miniaturized thermocycler.

FIG. 19 Shows a droplet arrangement (see also FIG. 18 h) according to an embodiment of the invention. (PCB) printed circuit board; (G) perfluorinated glass substrate; (M) permanent magnet; (T) [one of four donut-shaped] miniaturized thermocycler(s); (Sa) 100 μL raw sample solution containing 24.5 μL throat swab sample spiked with in vitro transcribed HPAI H5N1 RNA, 63.7 μL Lysis/Binding Solution, 6.9 μL water, 10 μL Lysis/Binding Enhancer, and 100 nL MagPrep Silica Particles; (W1) and (W2) Washing solution 1 and 2; (R) RT-PCR mixture covered by mineral oil. Scale bar, 10 mm.

FIG. 20 Shows on-chip nucleic acid amplification according to an embodiment of the invention. The C_{T} values of seven separate experiments were plotted versus the log of the number of HPAI H5N1 RNA copies. The solid black circles are the C_{T} values obtained after the droplet-based SPE; the dashed line (----) is a linear regression fit (R²=0.971) to the C_{T} values; the solid lines denote the upper and lower 95% confidence limits. The PCR efficiency (E) was 99%, E=10^{(-1/slope)}−1. 0.78-50 in vitro transcribed HPAI H5N1 RNA copies in 50 μL RT-PCR master mixture were used. In this embodiment, the real-time quantitative PCR (qRT-PCR) was performed off-chip using a DNA Engine Opticon™ 2 thermocycler (MJ Research).

FIG. 21 Shows a comparison of conventional and droplet-based SPE according to an embodiment of the invention. The QIAmp™ Viral RNA Mini Kit (QIAGEN) requires 140 μL raw sample as starting material. This is purified by adsorption to a silica membrane and preconcentrated into a final volume of 60 μL, of which 5 μL is added to a 45 μL RT-PCR master mixture. In this particular case, a volume of 5 μL prepurified RNA/RT-PCR is equivalent to 24.5 μL (18%) raw sample. Thus, 24.5 μL raw sample is the minimum volume a miniaturized thermocycler has to process to rival a conventional one. In this experiment, 140 μL of a 164.5 μL throat swab sample, spiked with 1.2×10⁷ in vitro transcribed HPAI H5N1 RNA copies, was used for the conventional and the remaining 24.5 μL for the droplet-based SPE. The dashed line (----) represents the conventional SPE (C_{T}=17.5); the solid line shows the droplet-based SPE (C_{T}=17.1); the solid/dotted line is the negative no template control (NTC); the solid thin line denotes the threshold value. (AU) arbitrary units. In this embodiment, the qRT-PCR was performed off-chip using a DNA Engine Opticon 2 thermocycler (MJ Research).

FIG. 22 Shows an optimization of PCR conditions according to an embodiment of the invention. Only after the fluorescence signal reaches a plateau for a particular step, the temperature is changed to the next level. After optimization, the time required for a thermal cycle including ramping is 22 s. The total mass of the thermocycler is 36 mg enabling heating/cooling rates of +11.5° C. s⁻¹ and −5.6° C. s⁻¹, respectively. Although the passive cooling makes up of 27% of the overall PCR process time, preferably we abstain from active cooling to keep down the overall power consumption. The solid line represents the temperature of the heater, the dashed line (----) shows the fluorescence intensity within the droplet. (AU) arbitrary units.

FIG. 23 Shows a melting curve analysis of a PCR product according to an embodiment of the invention. For the melting curve analysis, the following conditions were used: 95° C. for 3 s, 56° C. for 12 s, and ramping from 45° C. to 95° C. at 1° C. s⁻¹. The raw data was smoothened by a 5 point moving average using OriginPro™ 7 (OriginLab). The solid line represents the PCR product with a melting temperature of 76.0° C.; the dashed line is the negative control (NTC). (AU) arbitrary units. 5 reisolated in vitro transcribed HPAI H5N1 RNA copies were used as input.

FIG. 24 Shows a capillary electrophoresis (CE) of a PCR product according to an embodiment of the invention. (M1) marker 15 bp; (M2) marker 600 bp; (P) PCR product 114 bp; (AU) arbitrary units. The solid line represents the PCR product; the dashed line (----) is the negative control (NTC). The yield of the PCR product was 1.4 ng μL⁻¹. No primer dimer was visible. 5 reisolated in vitro transcribed HPAI H5N1 RNA copies were used as input.

FIG. 25 Shows an on-chip real-time PCR (qPCR) according to an embodiment of the invention. The CT values of seven separate experiments were plotted versus the log of the number of HPAI H5N1 cDNA copies. The solid black circles are CT values; the dashed line (----) is a linear regression fit (R²=0.999) to the CT values; the solid lines denote the upper and lower 95% confidence limits. The PCR efficiency (E) was 93%, E=10^{(-1/slope)}−1.12-1.2×10⁷ copies HPAI H5N1 cDNA in 500 nL qPCR mixture were used.

FIG. 26 Shows an overall process time according to an embodiment of the invention. Starting with a raw throat swab sample, it took less than 28 min to complete the fully automated process. The single process times (s) were: lysis/SPE (300 s), 4 times washing (40 s), RT (180 s), hot start (30 s), PCR including melting curve analysis (1,100 s). The solid line represents the RNA sample; the dashed/dotted line is the negative control (NTC); the solid thin line denotes the threshold value. (AU) arbitrary units. 500 in vitro transcribed HPAI H5N1 RNA copies were used as input.

FIG. 27 Shows a schematic of a prototype instrument according to an embodiment of the invention. In this embodiment, we have a stack of dedicated PCBs for the power supply (1), PC interface/single chip controller (2), thermal (3), optical (4), and microfluidic management (5) as well as a LCC68 carrier (6) for the PCR chip. A PC or a touch-screen display functions as user interface. Shown are the two topmost PCBs 5 and 6. A permanent magnet fixed on the spindle stepper motor of a CD-ROM drive remotely guides the droplet containing superparamagnetic particles within the enclosed, single-use plastic cartridge placed in a slot above the miniaturized thermocycler. A heat sink made of an air gap around the thin-walled PCR chamber enables fast temperature transition rates. The optics of the CD-ROM drive is replaced by an optical detection system with a modulated LED/demodulated photodetector, which allows real-time measurements in ambient light. For comparison, a portable 24×TEAC internal slim IDE CD-ROM drive CD-224E is available for less than 100 US $. All process steps can be visually controlled. (Sp) spindle stepper motor; (C) cartridge; (D) detector; (F) focusing lens; (M) magnet; (T) miniaturized thermocycler, (Sa) sample lysis chamber, (W1) and (W2) washing chambers; (R) RT-PCR chamber.

### DETAILED DESCRIPTION OF THE INVENTION

The present invention provides a method and/or system which allows on-chip preconditioning of complex real-world samples and/or handling of limited amounts of target material and/or on-chip nucleic acid amplification process.

Droplet formation and position control; materials useful as droplet, magnetic attractable matter and/or chip surface constituents; resulting droplet and/or chip surface physico-chemical properties; and deposit of biological sample in the droplet are described elsewhere (e.g. WO 2007/094739) and are thus available to the person skilled in the art.

Often, but not necessarily, the sample will include, or will be expected to include, target matter or a precursor thereof. Such embodiments shall be illustrated by a number of examples: The target matter may for instance be a cell or a molecule added to or included in the sample, and it may be desired to obtain it in a purified or enriched form. As another example, the target matter may be a compound known or theorized to be obtainable from a precursor compound by means of a chemical process. In this case the sample may for instance include a solution of such a precursor compound. As further example, a cell culture media may be suspected to be contaminated. In this case, the method of the present invention may be used to identify the type of contaminant.

The target matter or precursor thereof may thus be of any nature. Examples include, but are not limited to, a nucleotide, an oligonucleotide, a polynucleotide, a nucleic acid, a peptide, a polypeptide, an amino acid, a protein, a synthetic polymer, a biochemical composition, a glycoprotein, a radioactive compound, a polyelectrolyte, a polycation, a polycatanion, a pathogen, an organic chemical composition, an inorganic chemical composition, a lipid, a carbohydrate, a combinatory chemistry product, a drug candidate molecule, a drug molecule, a drug metabolite, a cell, a virus, a microorganism or any combinations thereof. In embodiments where the target matter is for example a protein, a polypeptide, a peptide, a nucleic acid, a polynucleotide or an oligonucleotide, it may contain an affinity tag. Examples of affinity tags include, but are not limited to biotin, dinitrophenol or digoxigenin. Where the target matter is a protein, a polypeptide, or a peptide, further examples of an affinity tag include, but are not limited to, oligohistidine (such as a penta- or hexahistidine-tag), polyhistidine, a streptavidin binding tag such as the STREP-TAGS® described in US patent application US 2003/0083474, U.S. Pat. No. 5,506,121 or U.S. Pat. No. 6,103,493, an immunoglobulin domain, maltose-binding protein, glutathione-S-transferase (GST), calmodulin binding peptide (CBP), FLAG-peptide (e.g. of the sequence Asp-Tyr-Lys-Asp-Asp-Asp-Asp-Lys-Gly) [SEQ ID NO: 1], the T7 epitope (Ala-Ser-Met-Thr-Gly-Gly-Gln-Gln-Met-Gly) [SEQ ID NO: 2], maltose binding protein (MBP), the HSV epitope of the sequence Gln-Pro-Glu-Leu-Ala-Pro-Glu-Asp-Pro-Glu-Asp [SEQ ID NO: 3] of herpes simplex virus glycoprotein D, the Vesicular Stomatitis Virus Glycoprotein (VSV-G) epitope of the sequence Tyr-Thr-Asp-Ile-Glu-Met-Asn-Arg-Leu-Gly-Lys [SEQ ID NO: 4], the hemagglutinin (HA) epitope of the sequence Tyr-Pro-Tyr-Asp-Val-Pro-Asp-Tyr-Ala [SEQ ID NO: 5] and the “myc” epitope of the transcription factor c-myc of the sequence Glu-Gln-Lys-Leu-Ile-Ser-Glu-Glu-Asp-Leu [SEQ ID NO: 6]. Where the target matter is a nucleic acid, a polynucleotide or an oligonucleotide, an affinity tag may furthermore be an oligonucleotide tag. Such an oligonucleotide tag may for instance be used to hybridize to an immobilized oligonucleotide with a complementary sequence. A respective affinity tag may be located within or attached to any part of the target matter. As an illustrative example, it may be operably fused to the amino terminus or to the carboxy terminus of any of the aforementioned exemplary proteins.

In one embodiment, the herein described method and/or system include nucleic acid amplification. When such embodiment is implemented, and in some particular embodiment, for convenience the word “purify” a nucleic acid molecule is used. One should understand that in some cases “purify” may mean to isolate, concentrate or enrich said nucleic acid molecule from a sample, a mixture and/or biological sample. In such context, “purify” should not be limited to 100% purification of said nucleic acid molecule. Rather, in this context, “purifying said nucleic acid molecule” may mean to isolate, concentrate or enrich cells comprising said nucleic acid molecule, or may mean to isolate, concentrate or enrich said nucleic acid molecule, from a mixture or biological sample, followed by a subsequent cell lysis and optionally, further purification, enrichment or isolation of said nucleic acid molecule from the cell lysate. In this context, “purifying said nucleic acid molecule” may also mean to isolate, concentrate or enrich said nucleic acid molecule, from a mixture or biological sample.

The foregoing is considered as illustrative only of the principles of the invention. Further, since numerous modifications and changes will readily occur to those skilled in the art, it is not desired to limit the invention to the exact examples and embodiments shown and described, and accordingly, all suitable modifications and equivalents may be resorted to, falling within the scope of the invention.

### Example I

### Materials and Methods

### Surface Chemistry

BB022022A1-glass microscope cover slips (Menzel) were sonicated in RBS 35-detergent (Pierce) at 50° C. for 10 min, rinsed with copious amounts of ultrapure water (Millipore) and redistilled isopropanol (J. T. Baker), and blown dry by nitrogen. After chemical vapor deposition of (heptadecafluoro-1,1,1,1-tetrahydrodecyl)trimethoxysilane (Gelest) at 150° C. and 100 Pa for 1 h, we spin coated the chemically modified glass substrates with a 1% solution of Teflon AF (DuPont) in FC-40 Fluorinert (3M) one to three times using the following program: 100 rpm s⁻¹, 500 rpm, 5 s, 300 rpm s⁻¹, 3000 rpm, 60 s. Hardbaking above 200° C. for 1 h produced 0.1-0.3 μm thick Teflon-like films with static contact angles with ultrapure water and M5904-mineral oil (SIGMA) of 115±2 and 85±2°, respectively.

### Microfluidics

For rotating the droplet containing the superparamagnetic particles, a stack of permanent N30H-neodynium iron boron disc magnet (ASSEMtech) was attached to a 0.9° Size 17 Super Slim Line-stepper motor (NetMotion) controlled by a K179-stepper driver (Ozitronics). The distance between the surface of the top magnet and droplet was around 0.8 mm, exposing the superparamagnetic particles to a magnetic field strength of around 0.4 T. To linearly move the droplet, we mounted the stepper motor to a ProScanII-motorized stage system (Prior Scientific). A program written in LabVIEW 8 software (National Instruments) served as user interface for the droplet manipulation.

Because of the smooth surface, there was no bubble formation at elevated temperatures and the droplet can be (over)heated to temperatures of up to 150° C. without bursting. If not stated otherwise, all reactions were carried out at room temperature (rt).

### Thermal Management

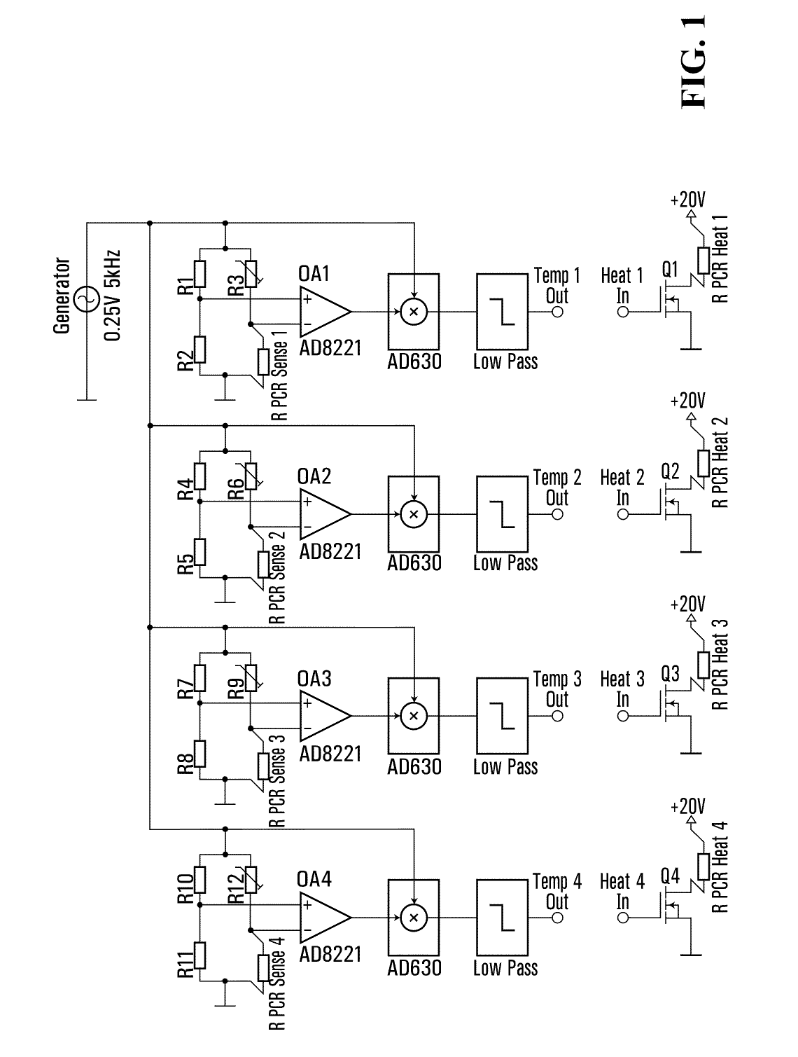

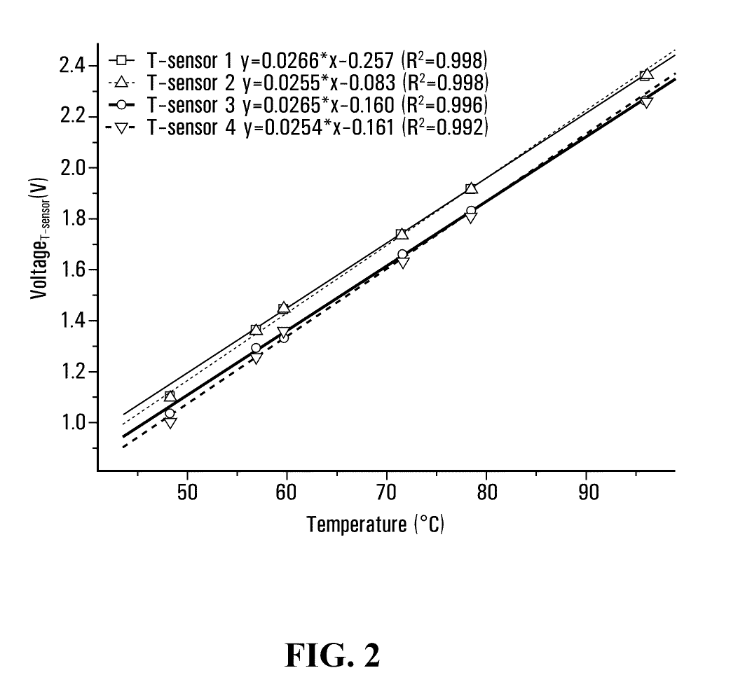

The thermal management of our silicon-micromachined PCR chip is described in detail elsewhere (Neuzil et al., Nucleic Acid Research, 2006, 34: 11, e77). However, in this particular embodiment, we numbered up the printed circuit board (PCB) comprising the thermal management for accommodating four individual heaters/temperature (T)-sensors 1-4 (FIGS. 1 and 2: H₁-H₄).

For fixing the Teflon-coated glass substrate on top of the four heaters/temperature sensors and improving the heat transfer into the corresponding temperature zones, it is preferable to apply small amount of mineral oil. The temperature distribution on the Teflon-coated glass substrate was measured using a PM200-IR camera (MTech Imaging) calibrated with a precision of ±0.2° C.

### Optical Detection

We continuously recorded the fluorescence signal into a text file using a BX-51-fluorescence microscope (Olympus), equipped with a X-Cite 120 PC-fluorescence illumination system (EXFO Life Sciences), 49002-filter set (CHROMA), H5784-20 photomultiplier tube (Hamamatsu), and TDS50054B-digital phosphor oscilloscope (Tektronix). The real-time data extracted thereof was the geometrical means of ten data points acquired during the last second of the extension.

### Cell Transfection

The THP-1 cells (ATCC) were transiently transfected with the pmaxGFP vector encoding the GFP using the Cell Line Nucleofector Kit V™ (Amaxa part of the Lonza group). After 24 h, the transfection efficiency was around 80%.

### Solid Phase Extraction (SPE)

We took the blood by finger-pricking from one of the inventors and stored it at 4° C. for 0-24 h in ethylenediaminetetraacetic acid (EDTA)-coated Microtainer-blood collection tubes (BD). Either reverse pipetting or wetting the inner and outer surfaces of the pipette tip with a thin film of M5904-mineral oil were used for metering the blood and suspension of the superparamagnetic particles.

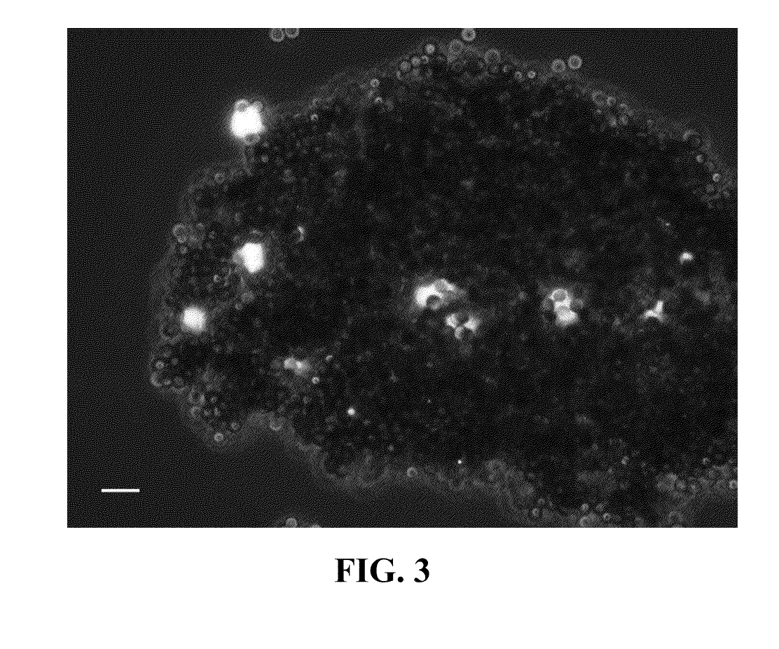

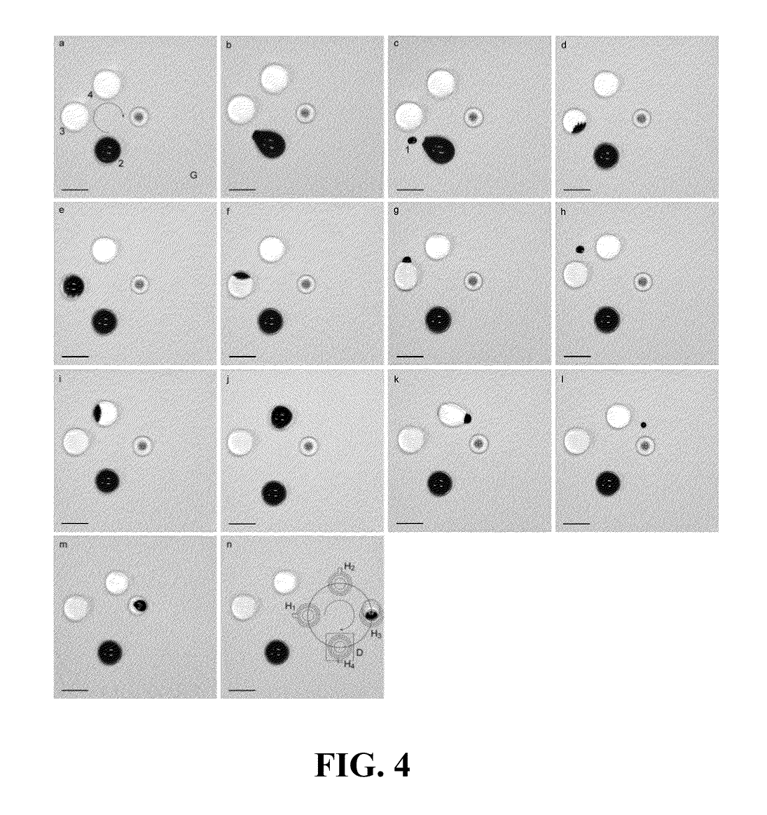

For the SPE, we added a 250 nL suspension of 400 μg/μL Dynabeads cluster of differentiation (CD)15 (Dynal Biotech) in 0.01 M phosphate buffered saline (PBS) (Sigma-Aldrich), pH 7.4/0.1% bovine serum albumin (BSA) (Roth) into a 25 μL blood droplet spiked with 30 GFP-transfected THP-1 cells, further mixed by ten times pipetting up/down, and incubated for 5 min. Thereafter, a 250 mL droplet containing the THP-1 cells immunocaptured onto the Dynabeads CD15 (FIG. 3) was split from the blood droplet and washed successively in two droplets containing 25 μL 0.01 M PBS/0.1% BSA (FIG. 4a-l).

It will be apparent to the person skilled in the art that any number of washes in any corresponding number of droplets may be suitable and fall within the present invention.

No fluorescent GFP-transfected THP-1 cells were any longer visible in the droplets containing the blood and the washing solutions. For manually counting the number of residual erythrocytes carried off into the washing solutions, we used a Bright-Light hemacyctometer (SIGMA).

### Cell Lysis

Then, the droplet containing the purified surface-immobilized THP-1 cells was merged with a droplet containing 1.5 μL PCR mixture (thus creating a “thermocycling droplet”) in the temperature zone 1 (corresponding to heater/T-sensor 1: H₁) and thermally lysed at 95° C. for 1 min to make their DNA accessible (FIG. 4m).

qPCR

The forward primer 5′-atg acc aac aag atg aag agc a-3′ [SEQ ID NO: 7] and reverse primer 5′-gta ggt gcc gaa gtg gta gaa g-3′ [SEQ ID NO: 8] amplified a 99 bp fragment of the transfected pmaxGFP™ vector (Amaxa part of the Lonza Group: the sequence of the pmaxGFP vector can be downloaded at the Amaxa/Lonza website), which was monitored in real-time employing the TaqMan probe 5′-FAM™-aag gcg ccc tga cct tca gcc cct a-3′-Eclipse Dark Quencher™ [SEQ ID NO: 9] (all of Research Biolabs). The PCR cocktail was based on the Taq PCR Core™ Kit (QIAGEN) and had the following composition: 28.0 μL water, 10.0 μL Q-Solution, 5.0 μL QIAGEN PCR Buffer, 1.0 μL dNTPs, 0.5 μL 10 μM TaqMan probe, 2.5 μL 10 μM primers each, and 0.5 μL Taq Polymerase. Of that, we used 1.5 μL for the miniaturized and the remainder for a bench-scale PCR on a DNA Engine Opticon 2 thermocycler (MJ Research). Non-transfected THP-1 cells served as negative template control (NTC). The PCR product specifity and yield was verified by capillary electrophoresis (CE) using a Bioanalyzer™ 2100 (Agilent).

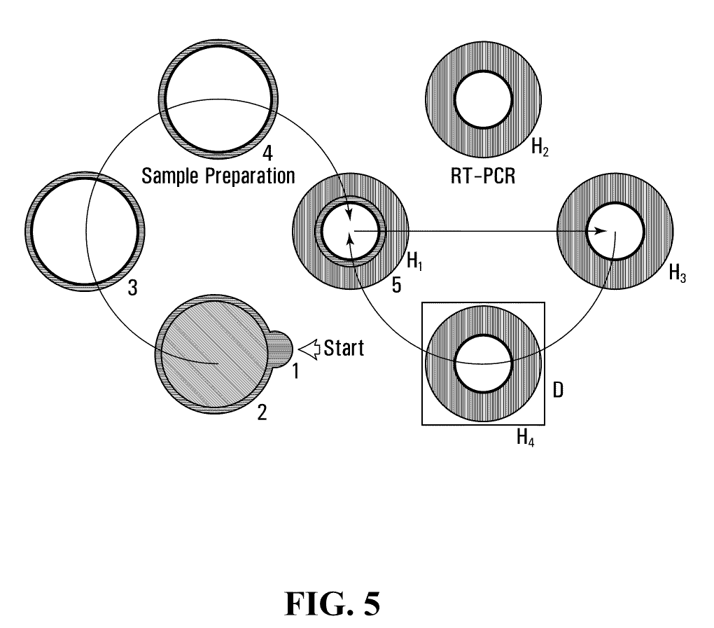

We ran the PCR by clockwise rotating the thermocycling droplet over the temperature zones 1-4 using the following two-step thermocycling protocols {Tₕₑₐₜₑᵣ[° C.] (t [s])}:
[1]—1 thermocycle of 95 (1), 723 (1), and 724 (1) (FIG. 5a) and 79 thermocycles of 95 (1), 602 (1), 723 (1), and 724 (1);
[2]—1 thermocycle of 95, (1), 603 (1), and 604 (1) (FIG. 5b) and 79 thermocycles of 95, (1), 602 (1), 603 (1), and 604 (1).

In both [1] and [2], one full rotation of the droplet corresponds to one thermocycle; and
[3]—1 thermocycle of 95 (1), 953 (1), and 604 (4) (FIG. 5c) and 79 thermocycles of 95, (1), 602 (4), 953 (1), and 604 (4).

In [3], except the first thermocycle, one full rotation of the droplet relates to two thermocycles.

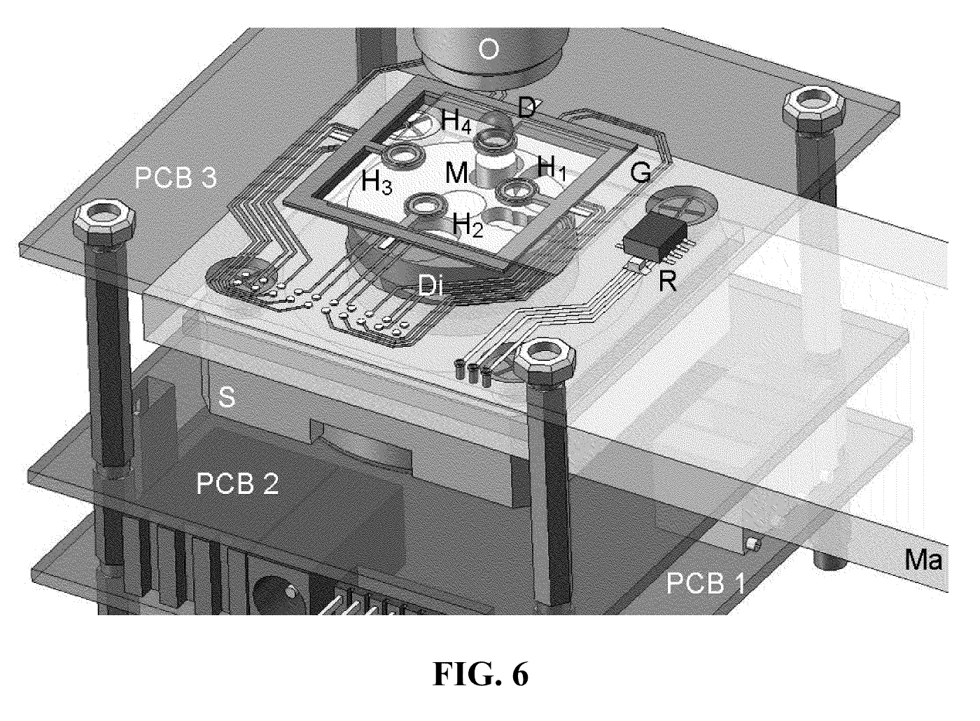

The angular velocity of the droplet during the PCR was 90° s⁻¹, which translates into 7-8 s for one thermocycle and overall reaction times of 560-640 s, respectively. 80 thermocycles were necessary for the fluorescence intensity to reach a plateau, but not to determine the threshold thermocycle (CT) that is generally the key diagnostic parameter for calculating a viral load. Therefore, 50 thermocycles with overall reaction times of 350-400 s, respectively, were more than sufficient. We placed the detector above the temperature zone 4 (corresponding to heater/T-sensor 4: H₄) for acquiring the real-time data during the last second of the extension step (FIG. 6).
[4] For optimization of the thermocycling conditions, we ran a two-step PCR in the time-domain by placing the droplet in temperature zone 4 (corresponding to heater/T-sensor 4: H₄). However, the TaqMan probe was replaced by 0.5 μL of 160,000-fold diluted SYBR Green I (Invitrogen) and the superparamagnetic particles were pulled out of the PCR solution after the thermal cell lysis.

### Results

### Droplet Formation

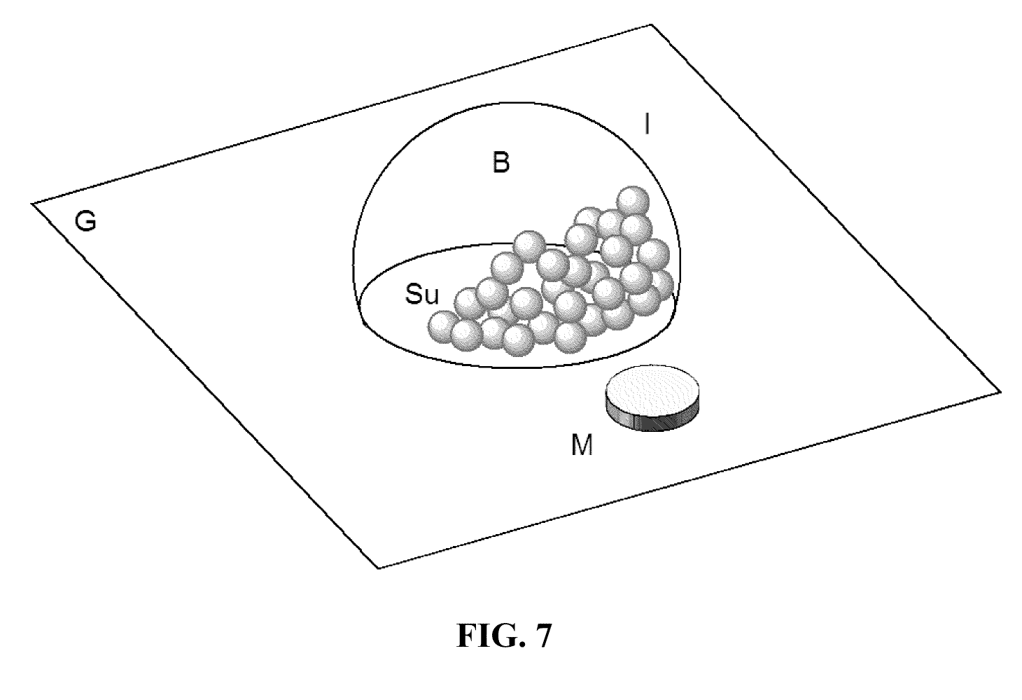

In one embodiment, a free 250 nL droplet spontaneously self-organizes on a Teflon-coated glass substrate by emulsifying an aqueous suspension of surface-functionalized superparamagnetic particles in an immiscible liquid (FIG. 7). Sealing the droplet, for instance, in mineral oil prevents its aqueous phase from evaporating and renders a complicated chip design for perpetuating a humidifying atmosphere unnecessary. The composition and concentration of a particular buffer solution remain unaltered—the prerequisite for performing (bio)chemistry in a droplet.

In one embodiment, adequate volume ratios of the aqueous phase and the mineral oil are about 100:1 for (bio)chemical processes at room temperature and about 1:5 for those requiring temperatures of up to 100° C. However, amplicons shorter than about 100 bp in length enable fast thermocycling and a volume ratio of about 1:3 is generally sufficient to account for evaporation. Other than mineral oil, long-chain alkanes with a boiling point above 150° C., silicone oil, or wax, and the like, e.g. as described in WO 2007/094739, are suitable for sealing the aqueous phase.

### Droplet Actuation

In one embodiment, an external permanent magnet is used for the droplet actuation. The magnetic field gradient exerts a translational force on the superparamagnetic particles suspended in the aqueous phase that, in turn, is transferred onto the inner aqueous phase/mineral oil interface. In one embodiment, to maximize the magnetic force, we raise the concentration of the superparamagnetic particles 40 fold.

Whether the droplet moves or splits, depends on a subtle balance between the magnetic force acting on the superparamagnetic particles, interfacial tension of the droplet, and friction force between the droplet and the Teflon-coated substrate surface. Given that, in one embodiment, the concentration of the superparamagnetic particles does not change over time, the magnetic force is constant, and the droplet moves, as long as the interfacial tension dominates over friction force. Because the hydrophilic surface of the exemplified anti-CD15-coated superparamagnetic particles makes them more affine towards the aqueous phase than to the surrounding mineral oil, they usually remain trapped inside the droplet. The droplet splits, as long as the friction force dominates over interfacial tension. After splitting with a dead volume close to zero, the aqueous suspension of superparamagnetic particles is still emulsified by a thin film of mineral oil. The outcome—moving or splitting—can easily be controlled by varying the volumes of interacting droplets: if their combined volume exceeds about 10 μL, a 250 nL droplet containing the superparamagnetic particles splits (FIG. 4).

### Droplets as Virtual Components of a μTAS

In one embodiment, besides being force mediators for actuating the droplet in a magnetic field, the surface-functionalized superparamagnetic particles temporarily serve as a solid support for (bio)chemical processes.

In one embodiment, placing the Teflon-coated glass substrate on micromachined heaters with integrated optics allows to follow temperature-controlled (bio)chemical processes in real-time. In this context, every droplet and/or droplet manipulation represents an item, equipment or task in a laboratory. For example, a droplet is like a tube (FIG. 4a), a droplet covered by mineral oil sitting on a microfabricated heater corresponds to a thermocycler (FIG. 4n), or surface-functionalized superparamagnetic particles with an immobilized biological sample passing through a droplet containing a washing solution resemble a filter (FIG. 4d-g), etc.

Components of the μTAS, illustrated in this embodiment, are virtual, which is why it is possible to realize a new prototype in minutes by implementing the teaching of the instant disclosure—simply a dispenser is needed for transferring a (bio)chemical protocol from a proven bench-scale into a droplet-based format.

In one embodiment, the magnetic force acting on the superparamagnetic particles is remote from the surface, therefore allowing using a single-use chip, run a dedicated test, and dispose the chip.

Solid Phase Extraction (SPE) of THP-1 Cells from Blood

Anticoagulants used in blood collection, RNases/DNases present in blood plasma and some leucocytes subtypes, and hemoglobin contained in erythrocytes significantly inhibit the PCR and have to be removed before. The ability of the anti-CD15-coated superparamagnetic particles to specifically isolate, preconcentrate, and purify the THP-1 cells expressing the CD15 cell surface marker from blood and pass them on to the PCR is key for performing sequential (bio)chemical processes.

On average, 25 μL of whole blood contains 2.5×10⁸ erythrocytes, 1.5×10⁷ platelets, 1.5×10⁵ leucocytes, 5×10⁴ lymphocytes, and 5×10³ NK cells. Of the leucocytes, the monocytes and granulocytes possess the CD15 cell surface marker and are therefore co-isolated under these conditions. However, the selectivity of the (bio)assay is determined later on by selecting PCR primers specific for the pmaxGFP™ transfection vector.

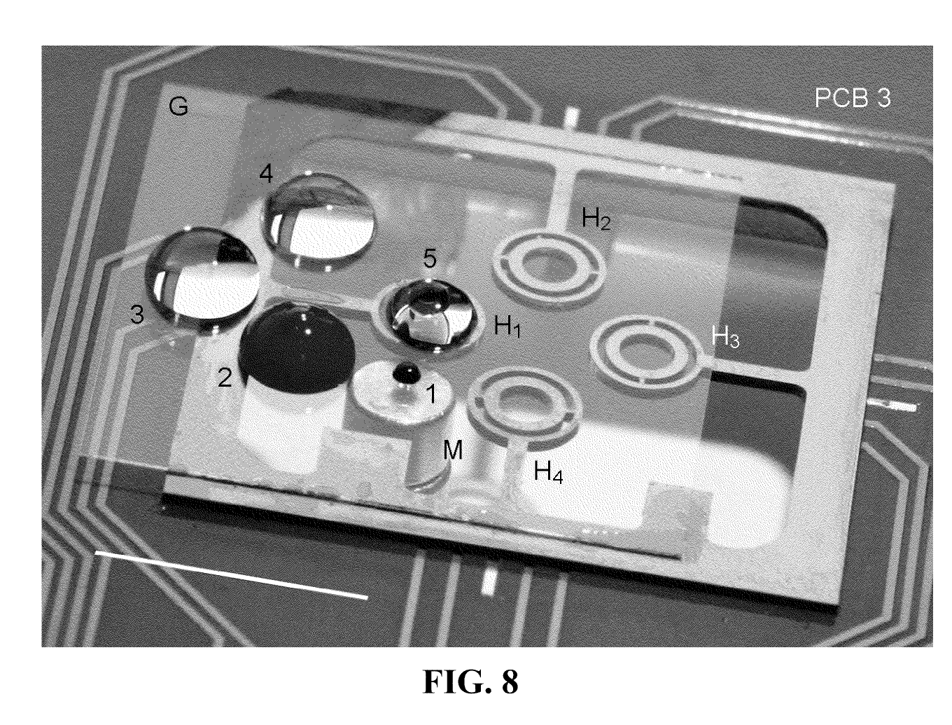

In one embodiment, consecutively, a 250 nL droplet holding the surface-immobilized THP-1 cells is split from a 25 μL blood droplet, purified in two 25 μL washing solution droplets, and merged with a 1.5 μL cell lysis/PCR mixture droplet (FIGS. 4 and 8). To optimize the recovery of the THP-1 cells and the parameters for washing and cell lysis, we use green fluorescent protein (GFP)-transfected THP-1 cells (FIG. 3). No GFP-transfected THP-1 cells are any longer observable in the blood and washing solutions (data not shown). Following the SPE, the GFP-transfected THP-1 cells transfer from a 25 μL to a 250 nL volume, corresponding to a 100-fold preconcentration. Potential PCR inhibitors like EDTA used for blood preservation, RNases/DNases, and erythrocytes largely stay behind in the blood or are diluted two times using the washing solution. Since the volume ratio of the superparamagnetic particles and the washing solution is 1:100, the contaminants possibly enclosed in the split droplet(s) are diluted at least 10⁴ [(1/100)²] fold. For example, on average 2.3×10⁴ out of 2.5×10⁸ erythrocytes present in a 25 μL blood volume are counted in the first [second] washing solution, representing a 10^{4[3]}-fold reduction (data not shown). We quantitatively recover 30 GFP-transfected THP-1 cells spiked into blood in generally less than 6 min with a purity suited for a PCR. The concentration ratio of the superparamagnetic particles and CD15-expressing cells of about 50:1 and relatively short diffusion path lengths between them enable a high yield and fast process time. The SPE of RNA/DNA or proteins from diverse body fluids, such as swabs, cell culture medium or urine works in a similar fashion.

### Cell Lysis in a Droplet

In one embodiment, after removing the PCR inhibitors, the surface-immobilized GFP-transfected THP-1 cells are thermally lysed within the PCR mixture located in the temperature zone 1 (FIGS. 4n and 8). Lysis at 95° C. for 1 min is generally sufficient for releasing the DNA from the GFP-transfected THP-1 cells into the PCR mixture. Higher temperatures of up to 130° C. and/or longer lysis times of up to 10 min do not affect the CT in the successive PCR targeting the GFP transfection vector (data not shown).

qPCR in the Space Domain in a Droplet

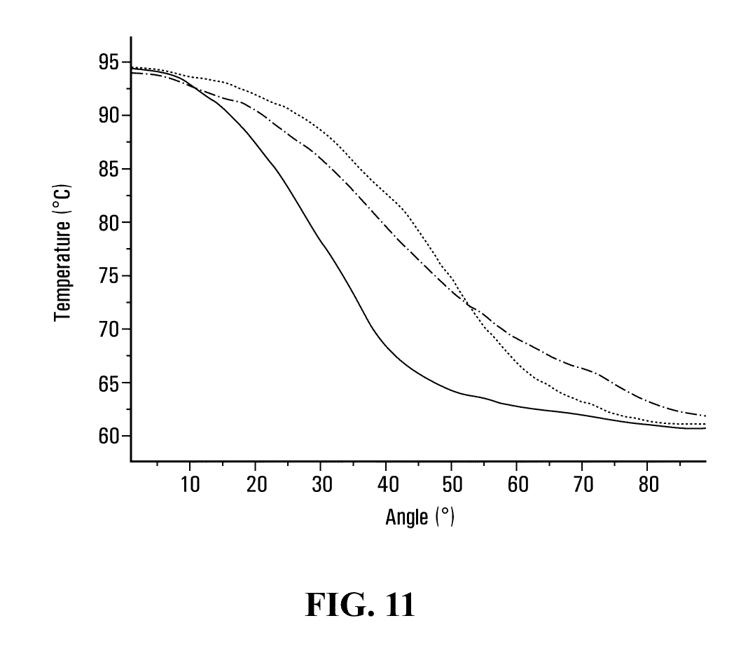

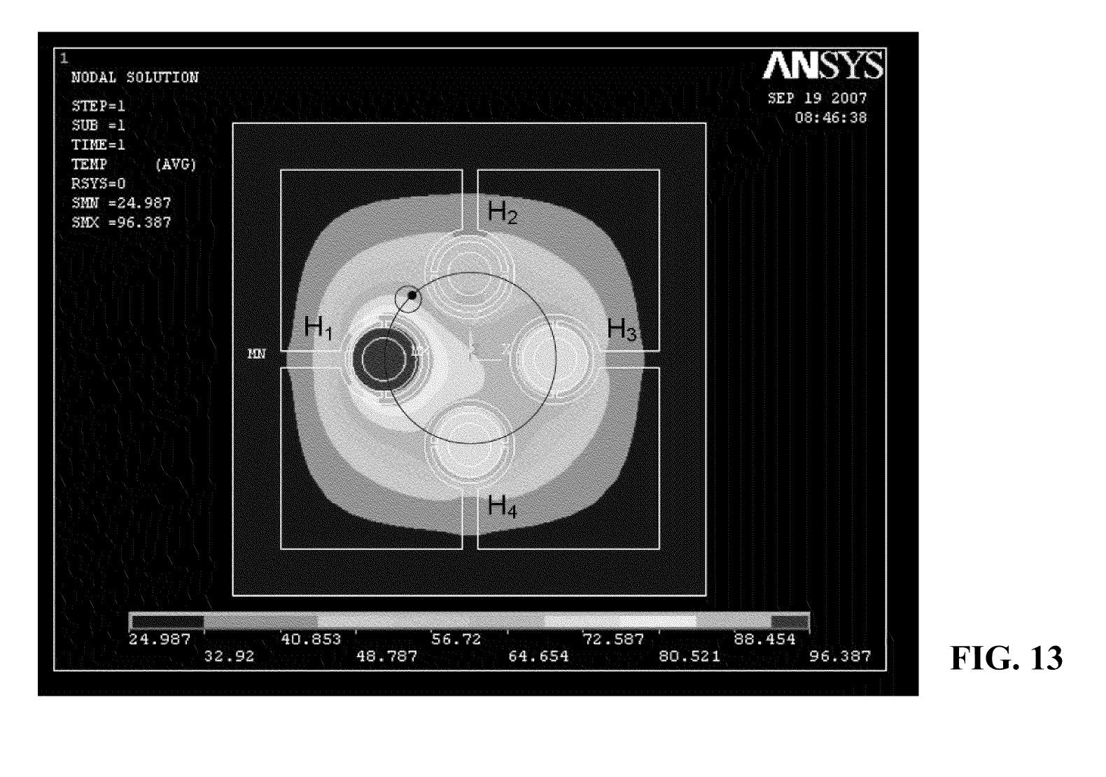

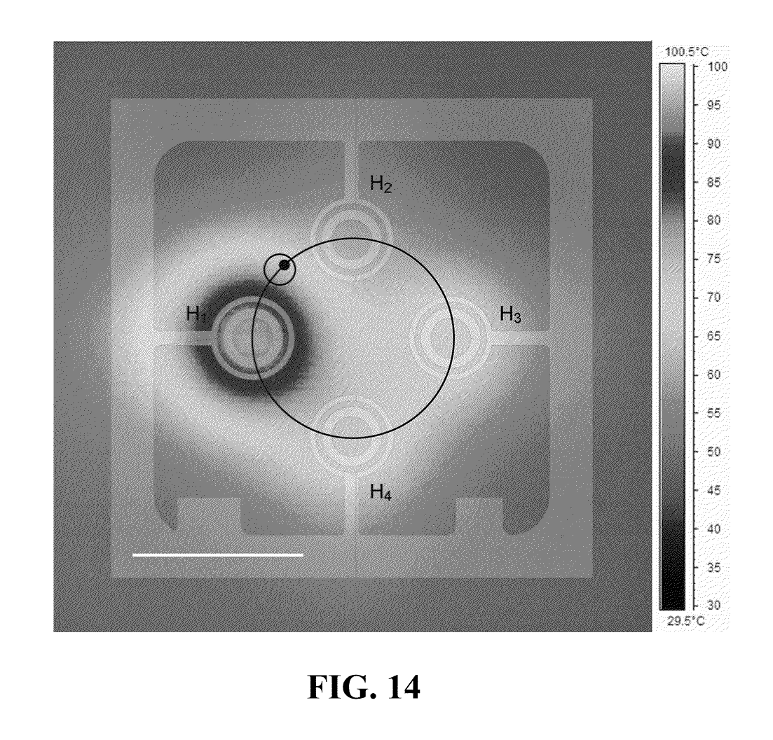

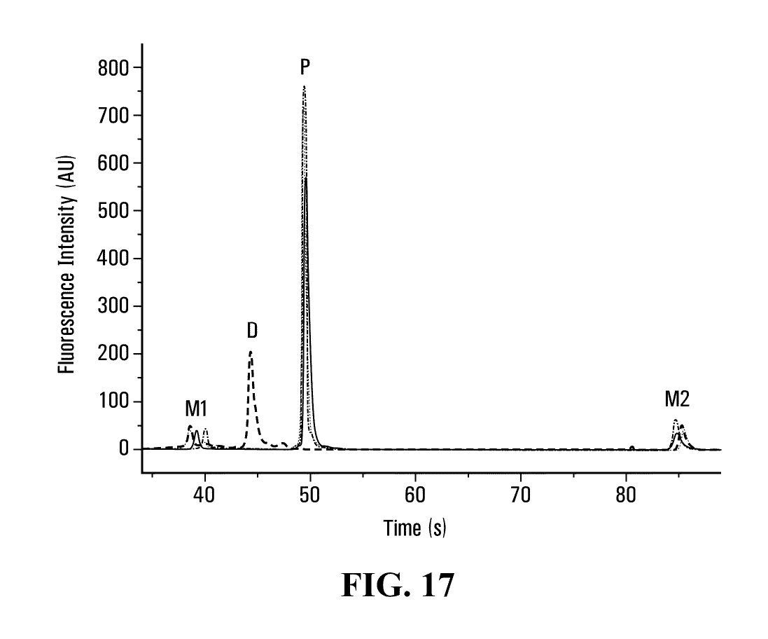

In one embodiment, starting from the temperature zone 1 (corresponding to heater/T-sensor 1: H₁), the droplet clockwise rotates over and pauses on the four temperature zones 1-4 (corresponding to heater/T-sensor 1-4: H₁-H₄) constantly maintained at two or three different temperatures (FIGS. 4n and 8). Since the relatively high concentration of the anti-CD15-coated superparamagnetic particles quench the fluorescence of intercalating dyes like SYBR Green I, Eva Green, or LC Green over time, this three-step thermocycling protocol (Supplementary FIGS. 11a, 13 and 14) is not preferred for a qPCR. However, the fluorescence quenching does not adversely affect the performance of the PCR itself (FIG. 17a). Alternatively, a TaqMan-based two-step thermocycling protocol is used for a qPCR.

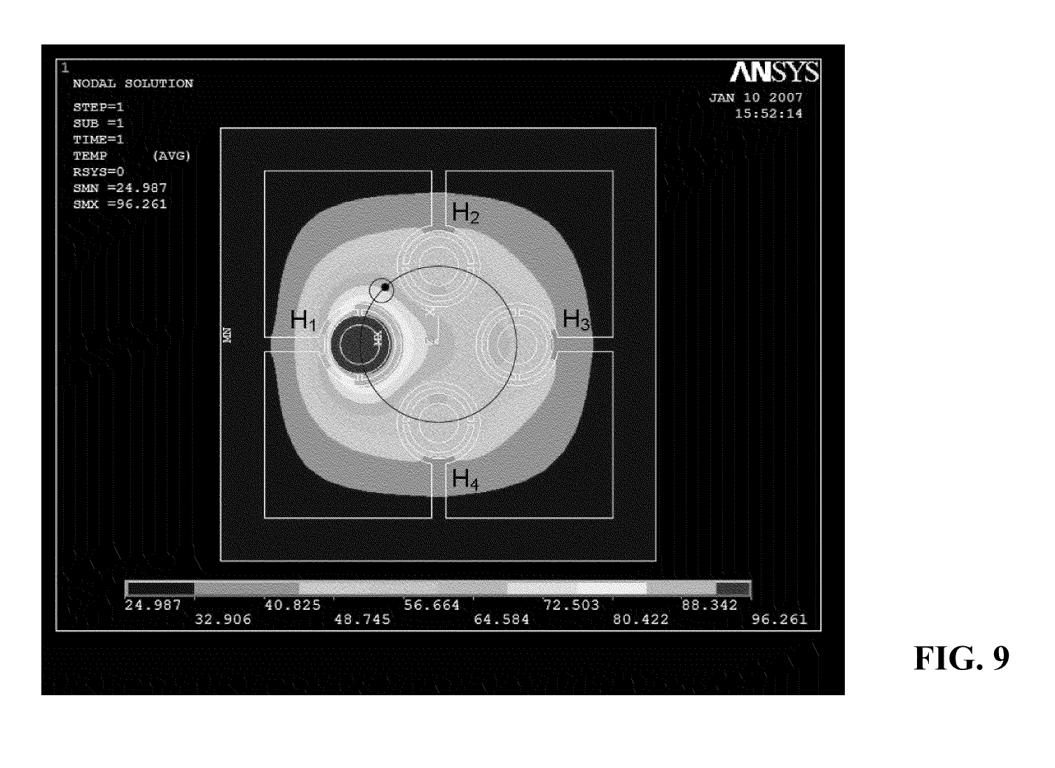

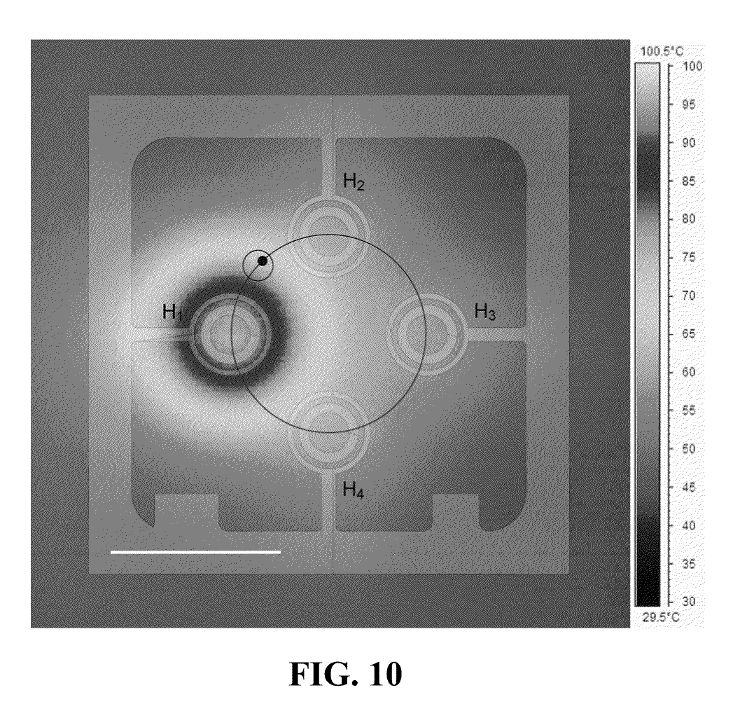

In one embodiment, for a two-step thermocycling protocol, we constantly keep the temperature zone 1 and the three temperature zones 2-4 at denaturation and annealing/extension temperatures, respectively. Stable temperatures for, homogeneous temperature distributions within, and a minimal thermal crosstalk between the four temperature zones 1-4 are preferable for the thermal layout of the PCR chip. The temperature variation measured by the temperature sensors is ±1° C. between room temperature (rt) and 95° C. Modeling using finite element analysis (FEA) (FIG. 9) and IR-imaging of the glass substrate surface (FIG. 10) show a temperature uniformity of ±1° C. between rt and 95° C. in the surface areas occupied by the droplet during the PCR. Large air gaps thermally isolating the four heaters/T-sensors from each other eliminate a thermal crosstalk between adjacent temperature zones on the glass substrate surface and result in a smooth temperature transition between them (FIG. 11b). Typically, the angular velocity during a rotation is 90° s⁻¹, correlating with heating and cooling rates of ±35° C. s⁻¹.

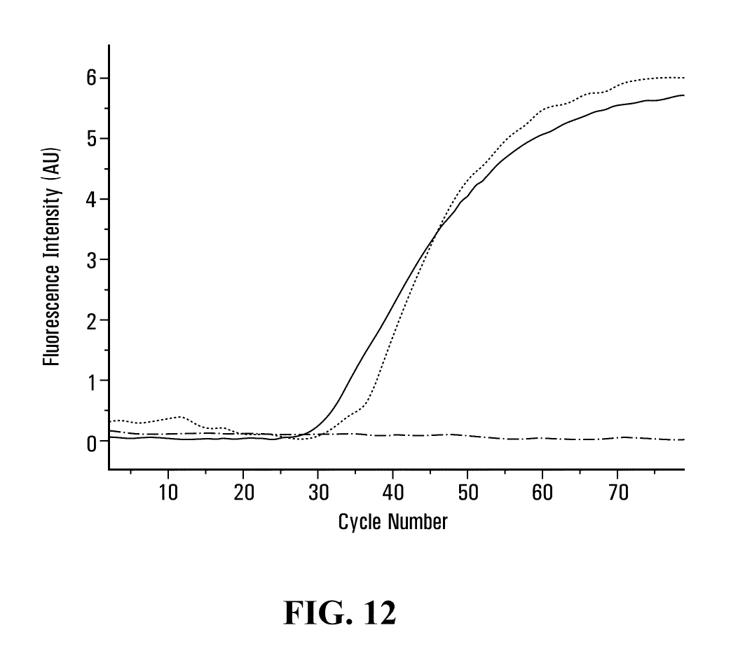

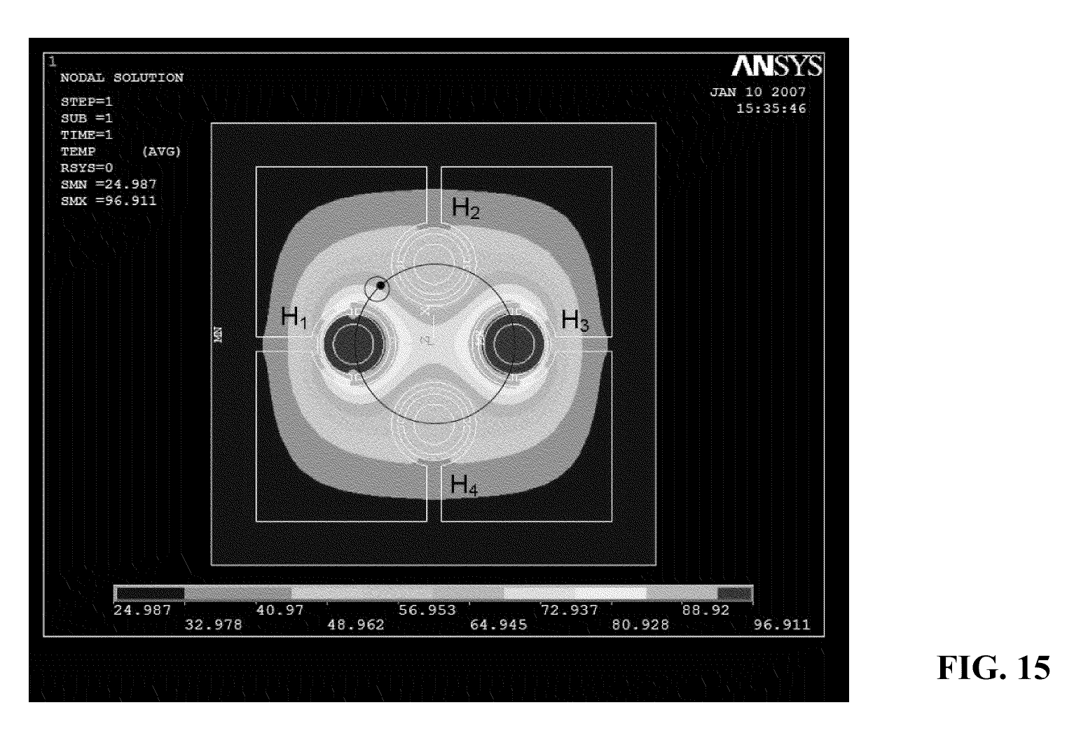

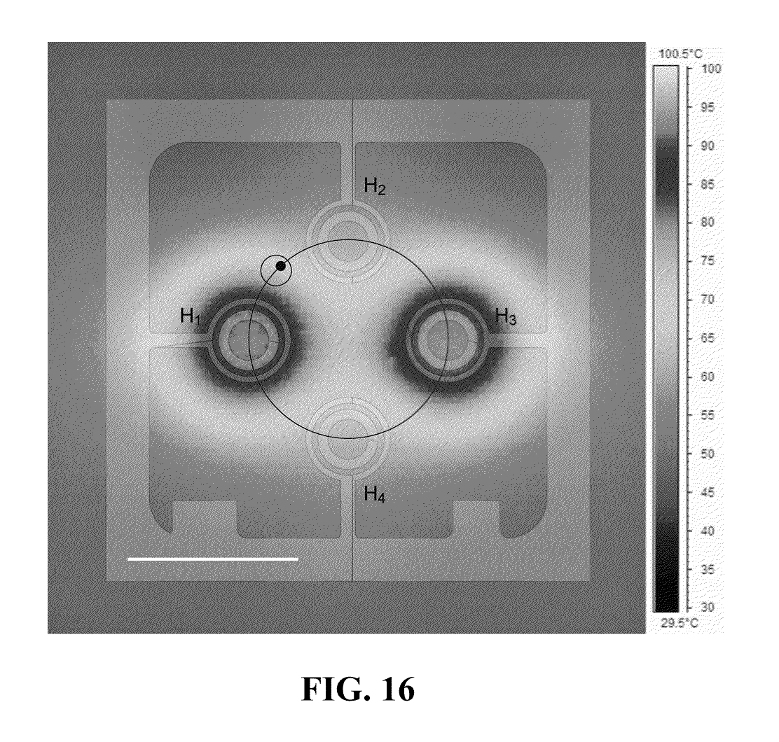

In one embodiment, a fluorescence microscope combined with a photomultiplier tube is placed above the temperature zone 4 (FIG. 6) and enables a qPCR (FIG. 12b). Running the qPCR in the time domain on the temperature zone 4 helps to shorten its run time, because it is possible to optimize the thermocycling conditions during one PCR experiment (Pipper et al., 2007, Nat. Med., 13, 1259-1263.). Including the transition times between adjacent temperature zones of 1 s each, we only need 8 s for one thermocycle. One thermocycle relates to one revolution, resulting in an overall PCR run time of 10 min 40 s for 80 thermocycles. Alternating the temperatures of successive temperature zones between 95 and 60° C. (FIGS. 11c, 15 and 16) further reduces the thermocycling time to 7 s. However, with two thermocycles per revolution, the resolution of the CT has an uncertainty of ±1 (FIG. 12c). To verify the PCR product specificity and yield capillary electrophoresis is used (FIG. 17). Even after 80 thermocycles no non-specifically amplified PCR products are observable. In this embodiment, a droplet volume of 1.5 μL yields about 30-40 ng/μL PCR product.

Except that, in this illustrative example, the relative concentration of the superparamagnetic particles is increased, we use established bench-scale protocols throughout the experiment. Without being bound to any theory, we believe that the overall reaction time of only 17 min is due to the short diffusion distances between the anti-CD15-coated surface of the superparamagnetic particles and the GFP-transfected THP-1 cells during the sample preparation together with the fast heat transfer intrinsic for a micro-scale PCR.

### Example II

### Methods and materials

### Surface Chemistry

In one embodiment, Teflon-like surfaces were obtained by spin-coating of VFM glass CoverSlips (CellPath) with a 1% solution of Teflon AF 1600 (DuPont) in FC-40 Fluorinert (3M). The films had a thickness of around 100 nm and the static contact angles with water and mineral oil (SIGMA) were 110±2° and 70±2°, respectively.

### Thermal Management.

The thermal management of our microfluidic device is described as in Example I. It is preferable to wet the microfabricated heater(s) with 100 nL of mineral oil to improve the thermal contact with the perfluorinated chip. Therefore, in this embodiment, we wetted the microfabricated heater(s) accordingly. Before use, every chip was sterilized at 130° C. for a few minutes on top of the microfluidic device.

### Optical Detection

In one embodiment, fluorescence was detected by a BX-51 Research Microscope (Olympus), equipped with a X-Cite 120 fluorescence illumination system (EXFO Life Sciences), a ET-OFF filter set (Ci-IROMA), a long-distance M Plan Apo 20× objective (Mitutoyo), and a H742I-40 photon counting module (Hamamatsu). We recorded the spectra continuously using a TDS50054B digital phosphor oscilloscope (Tektronix). Alternatively, we used our miniaturized optical detection system. Typically, the last 500 ms of the elongation step am used to acquire real-time data. If not stated otherwise, the raw data is used.

### Microfluidics

In one embodiment, the microfluidic device was mounted on a ProScanII-motorized stage system (Prior Scientific), which was moved relative to a permanent 117230357 neodymium iron boron disc magnet (ASSEMtech EUROPE) to manipulate the droplets containing superparamagnetic particles. We controlled the x,y-movement of the stage by LabVIEW 8 software (National Instruments).

### SPE of RNA

We used the MagMAX-96 Viral RNA Isolation Kit™ (Ambion). Due to the low magnetization of the original superparamagnetic particles, which makes them less preferable for some of the microfluidic manipulations, they were replaced by MagPrep Silica Particles™ (Merck) at a concentration of 200-500 μg/μL.

Because we did not have a biosafety level (BSL) 3 laboratory to work with the virus itself, in vitro transcribed HPAI H5N1 RNA of either the artus Influenza/H5 LC RT-PCR Kit (QIAGEN) or GIS and the cDNA thereof were used instead. A throat swab sample was taken front one of the inventors using the Viral CULTURETTE™ Collection and Transport System (BD).

For the SPE of the RNA, we added 100 nL of MagPrep Silica Particles to a droplet containing 24.5 μL throat swab sample spiked with in vitro transcribed HPAI H5N1 RNA, 63.7 μL Lysis/Binding Solution (Ambion), 6.9 μL water, and 10 μL Lysis/Binding Enhancer (Ambion), mixed for 10 s by pipetting up/down, and lysed for 5 min. Thereafter, the superparamagnetic particles were split from the sample droplet and washed successively for 10 s in two droplets containing 10 μL Washing Solution I (Ambion) and two droplets containing 10 μL Washing Solution 2 (Ambion). After the SPE, the RNA was desorbed from the superparamagnetic particles. It will be apparent to the person skilled in the art that any number of washes in any corresponding number of droplets may be suitable.

Depending on the RT-PCR kit, we used the following illustrative conditions (pH, T_{RT} [° C.], reaction time [s]): QuantiTect SYBR Green RT-PCR Kit (QIAGEN) (8.7, 50, 480), SuperScript III Platinum SYBR Green One-Step qRT-PCR™ Kit (Invitrogen) (8.4. 60, 180), and LightCycler RNA Master SYBR Green I™ Kit (Roche) (8.5, 61, 240).

qRT-PCR

We used the SuperScript III Platinum SYBR Green One-Step qRT-PCR mixture (Invitrogen) to monitor the RT-PCR in real-time (qRT-PCR) under the following illustrative conditions: RT at 60° C. for 180 s, initial activation at 95° C. for 20 s followed by 50 cycles of denaturation at 95° C. for 3 s, annealing at 56° C. for 12 s, and elongation at 72° C. for 7 s. To control the temperature within the droplet, the quantum yield of SYBR Green was recorded as a function of the temperature during the hot start phase. The PCR was followed by a melting curve analysis using the following conditions: 95° C. for 3 s, 56° C. for 12 s, and ramping from 45° C. to 95° C. at 1° C. s⁻¹.

In one embodiment, the 100 nL droplet containing the purified surface-bound RNA was moved onto a temperature zone located on top of the preheated microfabricated heater, merged with a droplet (thus creating a “thermocycling droplet”) containing 0.5 μL of the SuperScript III Platinum SYBR Green One-Step qRT-PCR mixture (nitrogen) and 2.5 μL mineral oil, mixed for 10 s, and subjected to the RT at a single location (temperature zone) on the chip surface.

We selected the primers using the HPAI A H5N1 virus A/chicken/Hubei/327/2004 (CKXF) strain, National Center for Biotechnology Information (NCBI) accession number AY684706, and aligned them to all H5N1 subtypes available in the NCBI Influenza Virus Resource as of October 2005.

The target was a 114 base pair (bp) fragment of the haemagglutinin (HA) segment of HPAI H5N1. We used 5′-CAA ACA GAT TAG TCC TTG CGA CTG-3′ [SEQ ID NO: 10] (HA114U.v1) and 5′-CYT GCC ATC CTC CCT CTA TAA A-3′ [SEQ ID NO: 11] (HA114L.v1) as forward and reverse primers, respectively.

For verifying the PCR product specificity and yield, we removed the magnetic particles and loaded the RT-PCR mixture into a DNA 500 LabChip Kit/Bioanalyzer™ 2100 (Agilent).

### Results

### Droplet Actuation

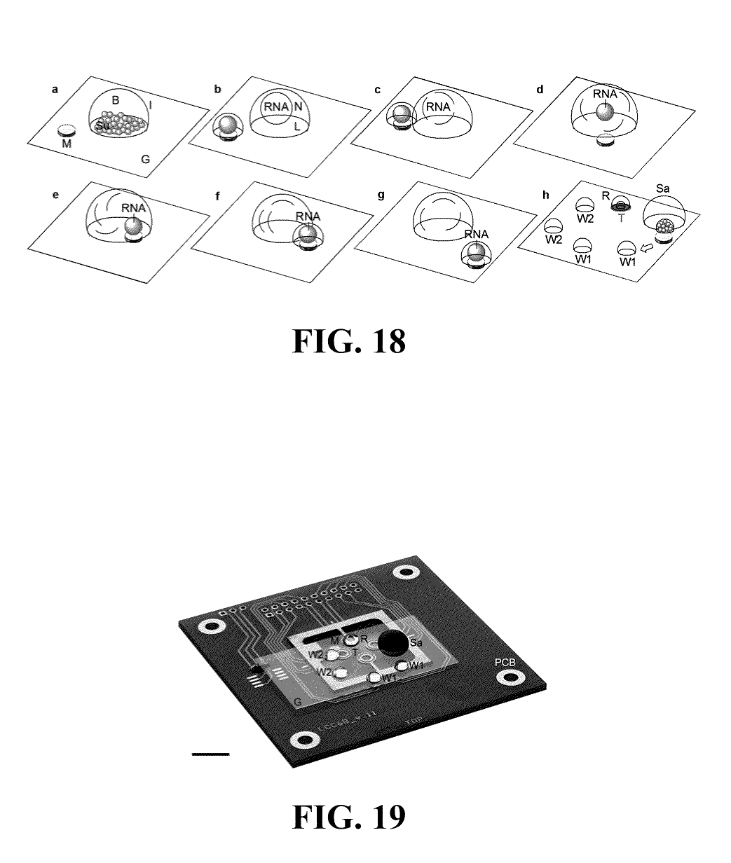

In one embodiment, a 100 nanoliter (nL) droplet spontaneously forms on a perfluorinated glass or polymer chip by emulsifying an aqueous suspension of surface-functionalized superparamagnetic particles in an immiscible liquid (FIG. 18a). In one embodiment, encapsulating the droplet in mineral oil both reduces the friction between the aqueous droplet and the solid surface and prevents the evaporation of the aqueous phase.

In one embodiment, the volume ratio of the aqueous phase and the mineral oil is about 100:1 for biochemical processes at room temperature and about 1:5 for those inquiring temperatures of up to 100° C. Similar to a biphasic segmented flow in microchannels, the aqueous phase is gliding on a thin film of mineral oil (Song, et al. Angew. Chem. Int. Edit (2006) 45, 7336-7356). In one embodiment, the thin film of mineral oil is preferred for handling highly viscous body fluids, such as a feces suspension, saliva, or whole blood.

In one embodiment, besides their role as force mediators to manipulate the droplet in a magnetic field (FIG. 18b-g), the superparamagnetic particles serve temporarily as a solid phase for biochemical processes (FIGS. 18h and 19). Pulling out the superparamagnetic particles from the aqueous droplet during the SPE requires their concentration to be raised by 1,000-2,500% in order to overcome the resistance imposed by the surface tension of the aqueous droplet.

In one embodiment, to perform temperature-controlled biochemical processes in real-time within a droplet, the chip is placed on at least one microfabricated heater with, optionally, an integrated optical detection system.

The shrinking of dimensions leads to increased surface-to-volume ratios (SVRs), which can cause biofouling induced by the non-specific adsorption of various components of the bioassay. This will not occur with a free aqueous droplet positioned on a hydrophobic surface, where only a relatively small fraction of its surface is in contact with the substrate. Interestingly, a miniaturized thermocycler composed of a 500 nL aqueous droplet has a smaller SVR (0.7 mm⁻¹) than 50 μL of water in a 200 μL PCR tube (1.5 mm⁻¹). In this context, the microfluidic system behaves like a macroscopic one and we do not undertake any additional measures to prevent biofouling.

### SPE of RNA

In one embodiment, the ability of the droplet to specifically retain or release the RNA after the SPE from the throat swab sample is key in performing the test in sequence, because only the surface-bound material is passed on to the RT-PCR. After washing away the contaminants, there are two alternatives to desorb the RNA from the superparamagnetic particles: decreasing the ionic strength of the solution by eluting in water or increasing the pH value and/or temperature of the solution. Elution first into water and then into the RT-PCR solution would involve an additional step. Since the reverse transcription (RT) is usually carried out at a pH value above 8.4 and at temperatures above 50° C., it is more convenient to directly release the immobilized RNA into the droplet containing the RT-PCR mixture at processing temperature. However, both procedures show no difference with respect to their cycle threshold (CT) in the subsequent RT-PCR targeting the haemagglutinin (HA) segment of HPAI H5N1 (data not shown).

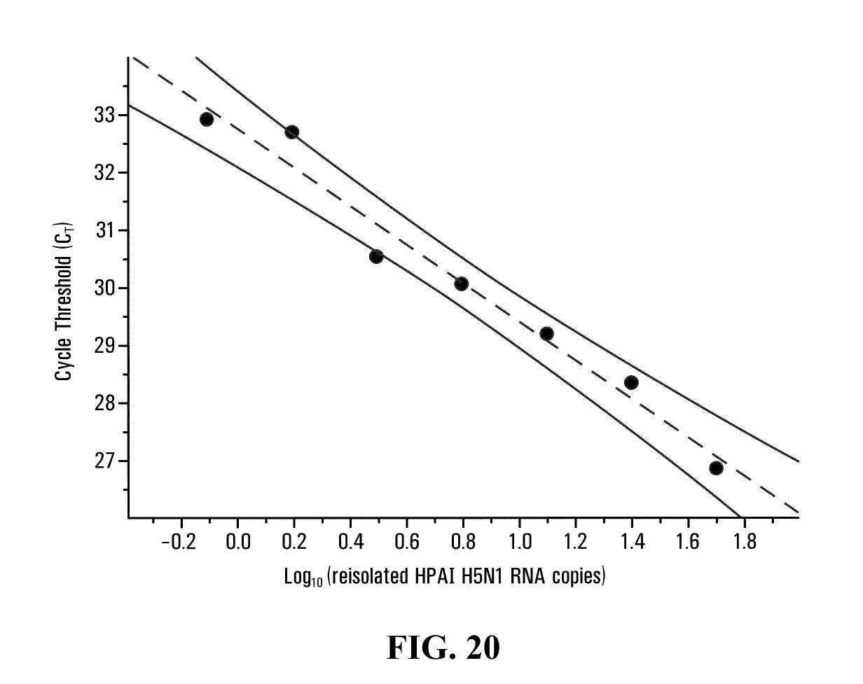

The recovery of 1-50 RNA copies from the throat swab sample using the droplet-based SPE is linear and shows an optimal PCR efficiency (FIG. 20), indicating highly pure reisolated RNA. Without being bound to any theory, we believe the high yield and purity is attributed to the fact that there is no loss of material due to non-specific adsorption to the substrate surface because the RNA remains adsorbed to the superparamagnetic particles during the SPE. Potential contaminants, which could interfere with the PCR, remain in the lysis/binding solution droplet (see FIG. 18). In addition, the surface-bound RNA is washed four times using 10 μL of washing solution. With a volume ratio of 1:100 for the superparamagnetic particles/washing solution, the remaining impurities are diluted 100 million [(1/100)⁴] times.

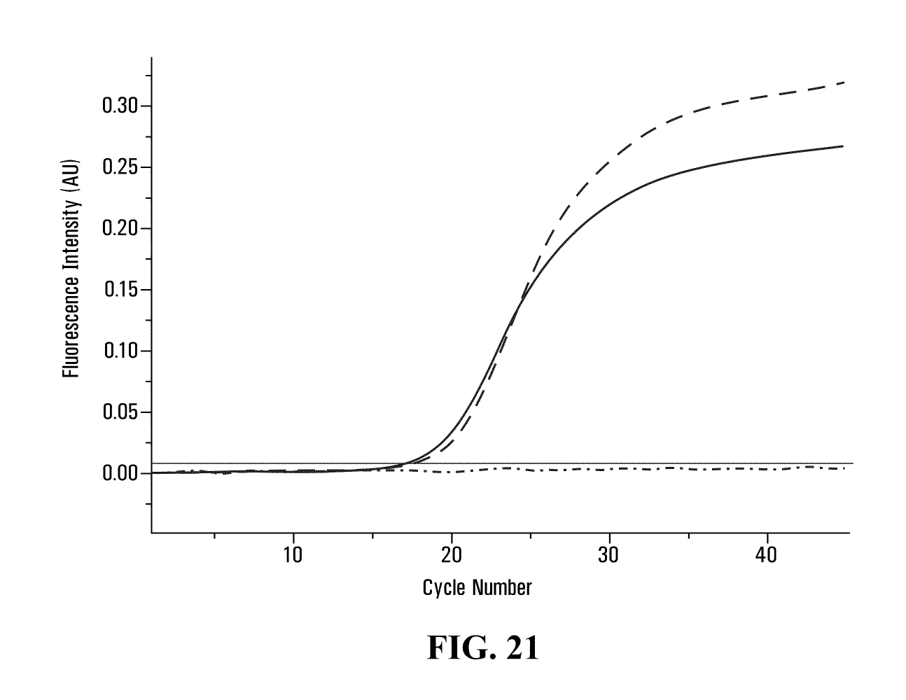

The ability to extract low copy numbers of RNA using a 100 nL suspension of superparamagnetic particles from a 100 μL raw sample volume corresponds to a preconcentration by 50,000%. This cannot be rivalled by any commercially available kit for the isolation of nucleic acids, which only use a fraction of the eluted material for the succeeding (q)RT-PCR (FIG. 21).

qRT-PCR

After SPE, the RNA is desorbed from the superparamagnetic particles into the 0.5-3 μL RT-PCR mixture at pH 8.4-8.7 and 50-61° C. over a period of 3-8 min. Depending on the type of RT-PCR kit that is used, the superparamagnetic particles are either removed or remain in the RT-PCR mixture before the hot start activation of the DNA polymerase.

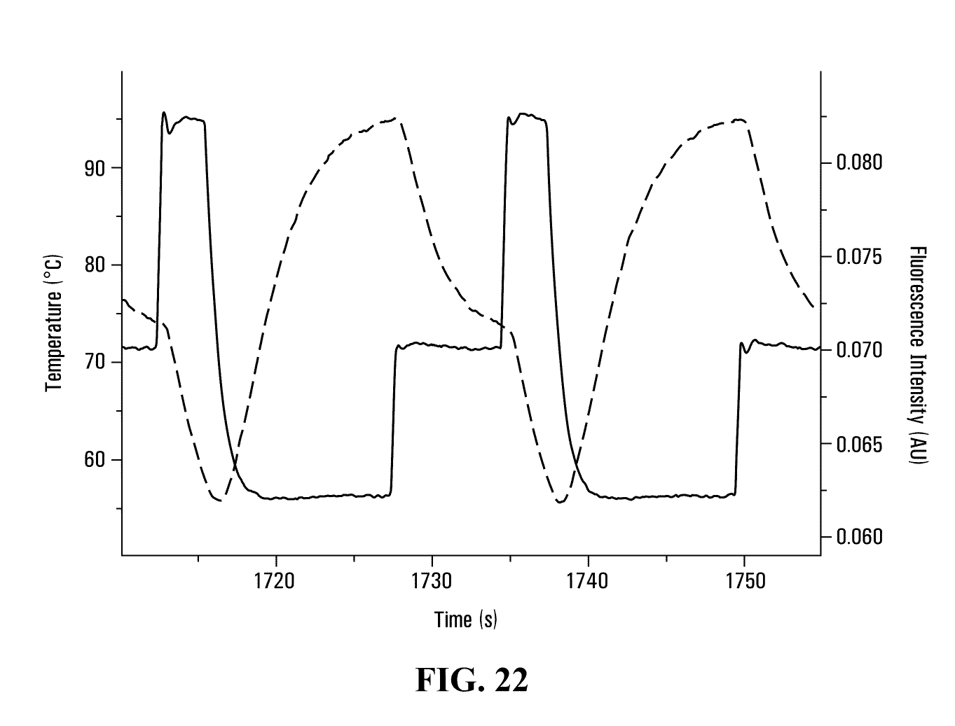

To minimize the qRT-PCR run time, each thermocycling step is shortened by decreasing holding times and/or increasing temperatures without compromising the PCR product specificity, yield, and efficiency. Since we continuously monitor the fluorescence signal and the temperature within the thermocycling droplet during the PCR, the thermocycling conditions can be optimized during one experiment (FIG. 22). Shorter thermocycles are preferable for reducing the thermal burden on the DNA polymerase. However, placing a disposable chip on top of the micromachined thermocycler more than outweighs the rather moderate ramping rates. The overall qRT-PCR run time employing a SYBR Green-based format with an antibody-modified hot start polymerase is 21 min 40 s. Using a two-step rather than a three-step thermocycling protocol and reducing the difference between consecutive temperatures would further shorten run times.

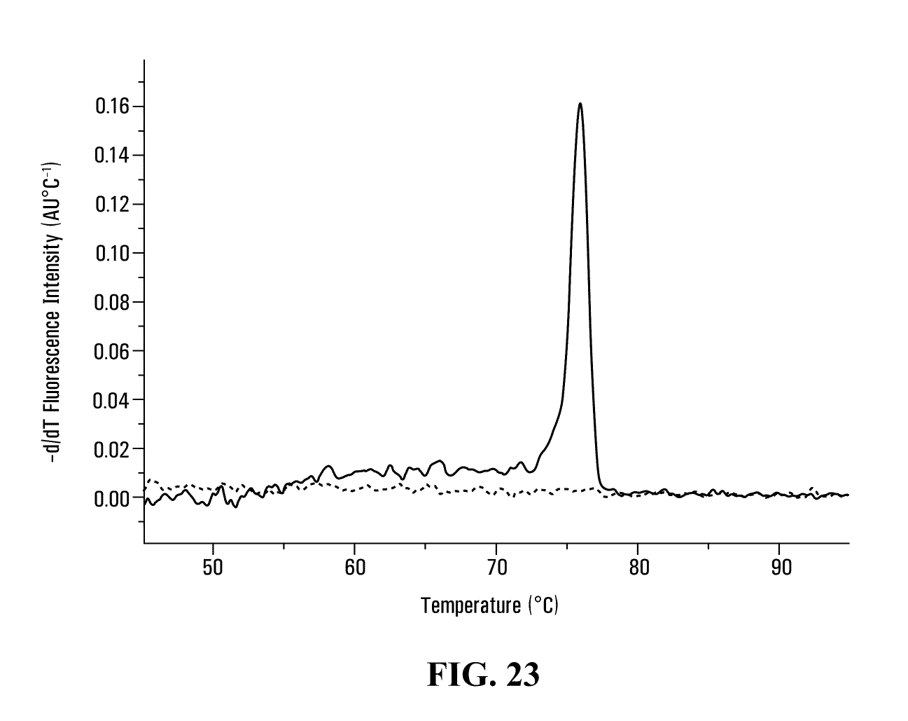

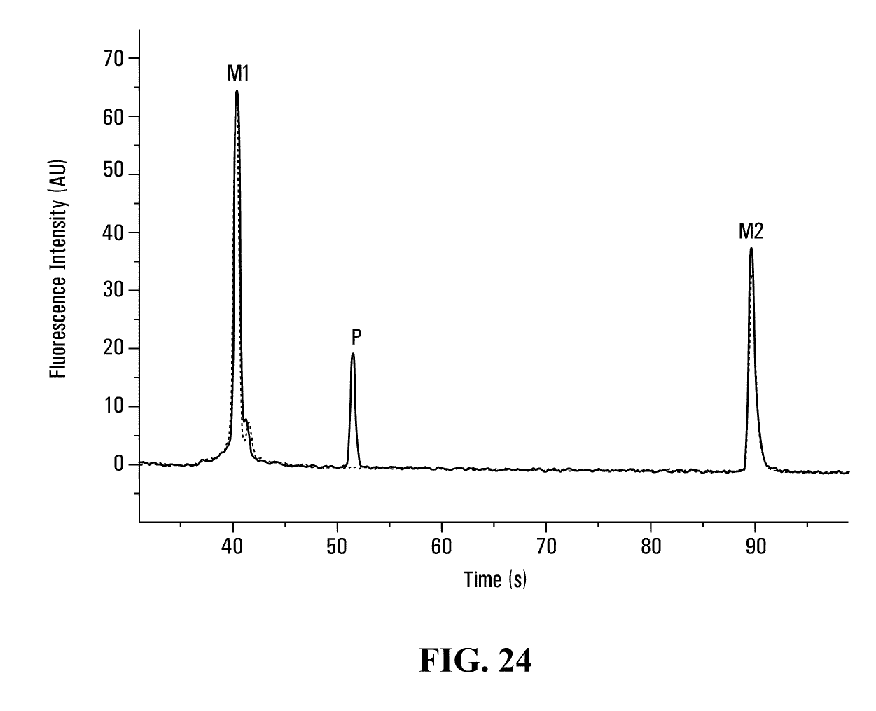

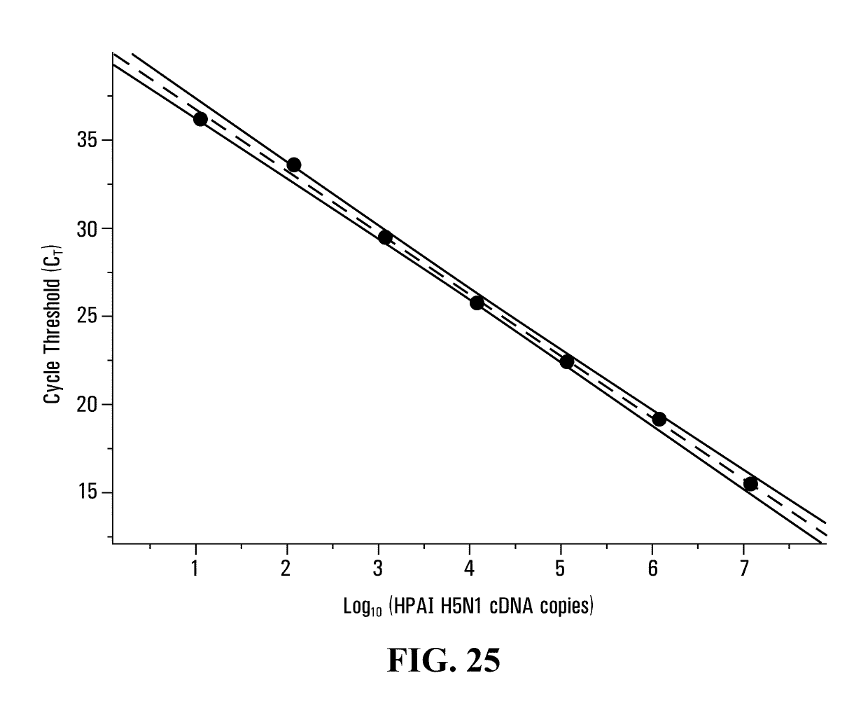

Melting curve analysis (FIG. 23) and capillary electrophoresis (CE) (FIG. 24) are used to verify the PCR product specificity and yield. Even after 50 PCR cycles, no non-specifically amplified PCR products are observed. Depending on the qRT-PCR volume, the PCR product yield is around 1-20 ng DNA μL⁻¹. The PCR efficiency over a dynamic range of six orders of magnitude is 93% (FIG. 25), whereby the correlation coefficient of 0.999 indicates an excellent linearity of the linear regression fit. If the qRT-PCR is carried out in 50 μL volumes using a conventional thermocycler, the PCR efficiency is close to 100% (data not shown). Without being bound to any particular theory, we hypothesize that the reduced PCR efficiency is due to the evaporation of water from the qRT-PCR buffer at elevated temperatures, thus causing the relative concentration of the qRT-PCR components to deviate from their optimal values. Since the qRT-PCR volume is only 500 nL, the cost is 2,000-5,000% lower compared to commercially available H5N1 tests, which typically rely on 20-50 μL/qRT-PCR.

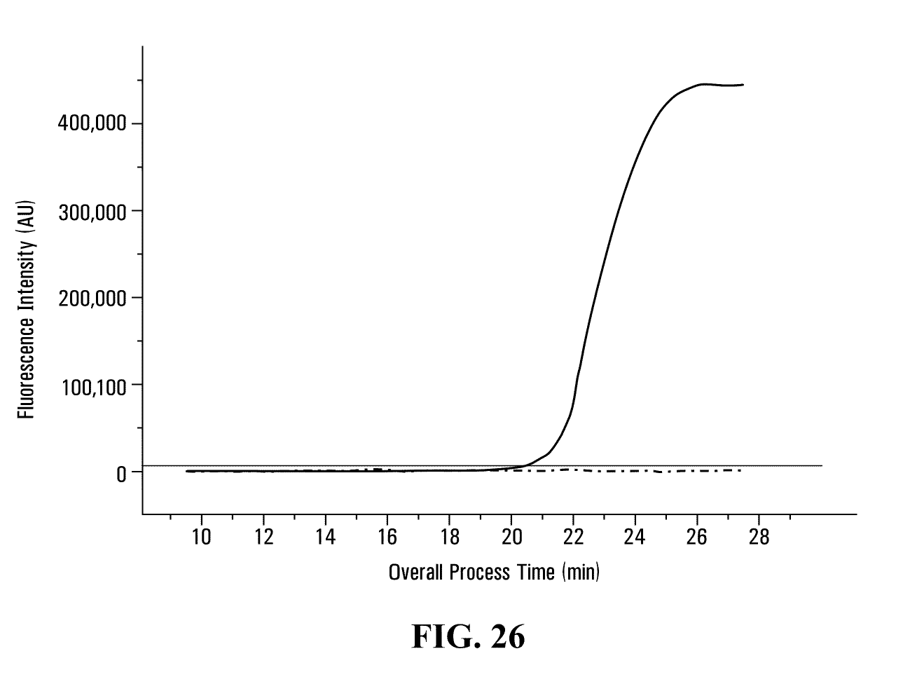

With the exception, in this illustrative example, of the concentration of the superparamagnetic particles, we follow established bench-top protocols for all single steps within the experiment. However, owing to the short diffusion distance between the surface of the superparamagnetic particles and the RNA during the SPE as well as the fast mass and heat transfer typical for a microsystem, the entire procedure is completed in less than 28 min (FIG. 26). This is about 440% faster than current H5N1 tests in the market that typically take around 4 h.

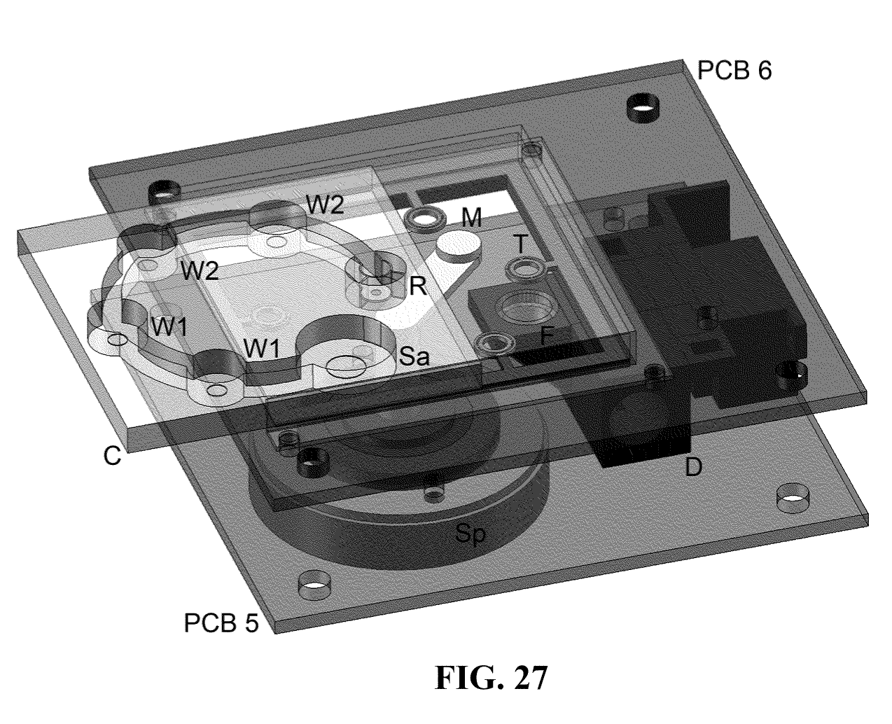

Point-of-care tests in low-resource settings demand low-cost, easy-to-use hand held units ideally composed of an instrument and a disposable. In one embodiment, there is provided a prototype instrument which, for the most part, relies on that of a CD-ROM drive (FIG. 27) utilizing its power supply, spindle stepper motor for microfluidic actuation, and optics for fluorescence detection. At present, we are replacing the macroscopic x, y—with the simple one-dimensional positioning system of a CD-ROM drive to allow for both linear and/or circular microfluidic actuation. Other work is targeting optical multiplexing by interrogating the rotating droplet at different locations between the temperature zones 2 and 4 using a miniaturized detector array working at different wavelengths.

The herein described strategies are not only applicable for these particular assays, but could easily be adapted for other assays, including but not limited to, detection of infectious diseases, such as but not limited to, SARS, HIV, hepatitis B/C, measles, tetanus, polio, tuberculosis, and the like, by extracting nucleic acid molecules, for instance, from body fluids, such as, but not limited to, blood sample, serum sample, urine sample, semen sample, plasma sample, lymphatic fluid sample, cerebrospinal fluid sample, naspharyngeal wash sample, sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumor sample, a tissue sample, a cell sample, a cell lysate sample, a virus culture sample, a forensic sample, an infection sample, a nosocomial infection sample, and the like apparent to those skilled in the art, or any combinations thereof.

Since there are an increasing number of (superpara)magnetic particle-based biochemical kits for the processing of cells, RNA, DNA, and proteins now commercially available, the microfluidic method and/or system described herein is an attractive diagnostic platform, especially for decentralized environmental, biological or medical testing.

From a conceptual perspective, past strategies to perform a (q)PCR in the space-domain have ignored fast temperature changes within an individual temperature zone and rely on an inflexible microchannel-inspired chip design.

By contrast, the low thermal masses of the herein described heaters/T-sensors come along with fast temperature transitions within the corresponding temperature zones allowing, in one embodiment, impressing temperature gradients in at least one temperature zone between subsequent, or within the same, thermocycle(s).

Additionally, the variable residence times of the droplet in a given temperature zone permit, in one embodiment, to customize the denaturation, annealing and/or extension times within the same or between different PCR runs. For example, these unique features—incrementally in- or decreasing the temperatures and/or holding times during a PCR—enable running a touch-down PCR, prolonging the denaturation of genomic DNA in early thermocycles, activating different types of hot start DNA polymerases, implementing a prolonged final extension, etc.

Accordingly, in one embodiment, the herein described method and/or system allow amplification of a nucleic acid molecule whereby
the residence time can be independently controlled/varied at each temperature zone (heated location), for instance between neighboring temperature zones, within a single thermocycle and/or between subsequent thermocycles;
the temperature can be independently controlled/varied at each temperature zone (heated location), for instance between neighboring temperature zones, within a single thermocycle and/or between subsequent thermocycles;
the residence time gradients can be independently controlled/varied at each temperature zone (heated location), for instance between neighboring temperature zones, within a single thermocycle and/or between subsequent thermocycles;
the temperature gradients can be independently controlled/varied at each temperature zone (heated location), for instance between neighboring temperature zones, within a single thermocycle and/or between subsequent thermocycles; and/or
the superposition of residence time gradients and temperature gradients can be independently controlled/varied at each temperature zone (heated location), for instance between neighboring temperature zones, within a single thermocycle and/or between subsequent thermocycles.

Additionally, in one embodiment, the herein described method and/or system allow amplification of one or more nucleic acid molecules. The person skilled in the art will readily understand that more than one set of specific primers may be used in a single or in subsequent PCR runs, thus allowing amplification of said one or more nucleic acid molecules.

Additionally, in one embodiment, the herein described method and/or system allow real-time monitoring which can be accomplished with or without the presence of magnetic particles (after releasing the RNA/DNA into, for instance, the RT-PCR mixture, the magnetic particles may be split from the RT-PCR mixture; thereby, magnetic particles associated quenching effects can be avoided).

Although the present invention has been described in considerable detail with reference to certain preferred embodiments thereof, variations and refinements are possible without departing from the spirit of the invention. Therefore, the scope of the invention should be limited only by the appended claims and their equivalents.

All references cited throughout the specification are hereby incorporated by reference in their entirety.

We claim:

1. A method for amplifying a nucleic acid molecule, said method comprising:
(a) providing a fluid droplet, said fluid droplet comprising an inner phase and an outer phase,
wherein the outer phase is immiscible with the inner phase, and the outer phase is surrounding the inner phase, the inner phase comprises a sample comprising or suspected of comprising said nucleic acid molecule, the inner phase is shielded from the environment by the outer phase, and said inner phase comprises surface-functionalized magnetically attractable matter;
(b) providing at least one surface;
(c) providing at least a heater for heating a respective temperature zone on said at least one surface;
(d) disposing said fluid droplet onto said at least one surface; and
(e) processing said nucleic acid molecule on said at least one surface, said processing comprising (i) controlling the position of said magnetically attractable matter relative to said at least one surface so as to purify said nucleic acid molecule; and (ii) amplifying said nucleic acid molecule, said amplifying comprising locating said magnetically attractable matter onto said temperature zone.

2. The method of claim 1, wherein said at least one surface comprises one surface.

3. The method of claim 1, wherein the magnetically attractable matter is at least one magnetically attractable particle.

4. The method of claim 3, wherein the at least one magnetically attractable particle comprises diamagnetic particle, a ferromagnetic particle, a paramagnetic particle, a superparamagnetic particle, or any combinations thereof, and wherein said magnetically attractable particle is optionally bound to said nucleic acid molecule during said amplifying.

5. The method of claim 1, wherein controlling the position of said magnetically attractable matter relative to said at least one surface comprises exposing said magnetically attractable matter to a magnetic or an electromagnetic field.

6. The method of claim 5, wherein controlling the position of said magnetically attractable matter relative to said at least one surface further comprises moving the magnetically attractable matter by altering said magnetic or electromagnetic field, moving said at least one surface, or a combination thereof

7. The method of claim 6, wherein altering said magnetic field comprises altering the position of at least one magnet.

8. The method of claim 5, wherein controlling the position of said magnetically attractable matter further comprises moving said magnetically attractable matter by means of said magnetic or electro magnetic field.

9. The method of claim 8, comprising moving said magnetically attractable matter and fusing said fluid droplet with at least one wash droplet.

10. The method of claim 9, wherein moving said magnetically attractable matter comprises splitting said magnetically attractable matter from said fluid droplet, and fusing said magnetically attractable matter with said at least one wash droplet.

11. The method of claim 9, further comprising splitting said magnetically attractable matter from said at least one wash droplet, and fusing said magnetically attractable matter with a thermocycling droplet.

12. The method of claim 11, wherein said thermocycling droplet is located onto said temperature zone.

13. The method of claim 12, wherein said thermocycling droplet is moved onto at least a second temperature zone, wherein said temperature zones are located on the same surface, said method further comprising providing at least a second heater for heating said at least second temperature zone.

14. The method of claim 13, wherein said thermocycling droplet is moved onto at least a third temperature zone, wherein said temperature zones are located on the same surface, said method further comprising providing at least a third heater for heating said at least third temperature zone.

15. The method of claim 14, wherein said thermocycling droplet is moved back onto said first temperature zone.

16. The method of claim 14, wherein said thermocycling droplet is moved onto at least a fourth temperature zone, wherein said temperature zones are located on the same surface, said method further comprising providing at least a fourth heater for heating said at least fourth temperature zone.

17. The method of claim 16, wherein said thermocycling droplet is moved back onto said first temperature zone.

18. The method of claim 15, wherein said thermocycling droplet resides substantially onto each of said temperature zones for a respective predetermined time which is independently controlled.

19. The method of claim 17, wherein said thermocycling droplet resides onto each of said temperature zones for a respective predetermined time which is independently controlled.

20. The method of claim 18, wherein said thermocycling droplet resides onto at least one of said temperature zones for said predetermined time which is independently controlled to vary between at least two thermocycling droplet movements onto said at least one of said temperature zones.

21. The method of claim 19, wherein said thermocycling droplet resides onto at least one of said temperature zones for said predetermined time which is independently controlled to vary between at least two thermocycling droplet movements onto said at least one of said temperature zones.

22. The method of claim 15, wherein each of said temperature zones has a respective predetermined temperature which is independently controlled.

23. The method of claim 17, wherein each of said temperature zones has a respective predetermined temperature which is independently controlled.

24. The method of claim 22, wherein said predetermined temperature of at least one of said temperature zones is independently controlled to vary between at least two thermocycling droplet movements onto said at least one of said temperature zones.

25. The method of claim 23, wherein said predetermined temperature of at least one of said temperature zones is independently controlled to vary between at least two thermocycling droplet movements onto said at least one of said temperature zones.

26. The method of claim 15, wherein at least one of said temperature zones has a temperature which is controlled to vary in time.

27. The method of claim 17, wherein at least one of said temperature zones has a temperature which is controlled to vary in time.

28. The method of claim 15, wherein said thermocycling droplet resides onto each of said temperature zones for a respective predetermined time which is independently controlled, and wherein each of said temperature zones has a respective predetermined temperature which is independently controlled.

29. The method of claim 17, wherein said thermocycling droplet resides onto each of said temperature zones for a respective predetermined time which is independently controlled, and wherein each of said temperature zones has a respective predetermined temperature which is independently controlled.

30. The method of claim 28, wherein said thermocycling droplet resides onto at least one of said temperature zones for said predetermined time which is independently controlled to vary between at least two thermocycling droplet movements onto said at least one of said temperature zones, and wherein said predetermined temperature of at least one of said temperature zones is independently controlled to vary between at least two thermocycling droplet movements onto said at least one of said temperature zones.

31. The method of claim 29, wherein said thermocycling droplet resides onto at least one of said temperature zones for said predetermined time which is independently controlled to vary between at least two thermocycling droplet movements onto said at least one of said temperature zones, and wherein said predetermined temperature of at least one of said temperature zones is independently controlled to vary between at least two thermocycling droplet movements onto said at least one of said temperature zones.

32. The method of claim 1, further comprising providing means to detect fluorescence.

33. The method of claim 32, wherein the means to detect fluorescence are for detecting fluorescence when said magnetically attractable matter is located substantially onto said temperature zone.

34. The method of claim 1, wherein said heater comprises Platinum or Silicon.

35. The method of claim 1, wherein said nucleic acid amplification comprises a reverse-transcriptase (RT), polymerase chain reaction (PCR), RT-PCR, a real-time quantitative PCR (qPCR), real-time quantitative RT-PCR (qRT-PCR), helicase dependent amplidication (tHDA), smart amplification process (SMAP), loop-mediated amplification (LAMP), rolling circle amplification (RCA), or recombinase polymerase amplification (RPA).

36. The method of any one of claim 1, wherein the fluid of said inner phase of the first fluid droplet is a polar liquid, and said surface is a non-polar surface.

37. The method of claim 36, wherein the outer phase of the first fluid droplet is a non-polar liquid.

38. The method of claim 36, wherein the fluid of said inner phase is water, deuterium oxide, tritium oxide, an alcohol, an organic acid, an inorganic acid, an ester of an organic acid, an ester of an inorganic acid, an ether, an amine, an amide, a nitrile, a ketone, an ionic detergent, a non-ionic detergent, carbon dioxide, dimethyl sulfone, dimethyl sulfoxide, a thiol, a disulfide, or a polar ionic liquid.

39. The method of claim 36, wherein the fluid of the outer phase is a mineral oil, a silicone oil, a natural oil, a perfluorinated carbon liquid, a partially halogenated carbon liquid, an alkane, an alkene, an alkine, an aromatic compound, carbon disulfide, or a non-polar ionic liquid.

40. The method of claim 36, wherein the non-polar surface is silicone, plastic, surface-modified silicon oxide, surface-modified silicon hydride, surface-modified paper, surface-modified glass, surface-modified quartz, surface-modified glimmer, surface-modified metal, surface-modified metal oxide, surface-modified ceramic, or any composites thereof

41. The method of claim 1, wherein the fluid of said inner phase of the fluid droplet is a non-polar liquid and said surface is a polar surface

42. The method of claim 1, wherein the sample is from a human.

43. The method of claim 1, wherein the sample is selected from the group consisting of a blood sample, serum sample, urine sample, semen sample, plasma sample, lymphatic fluid sample, cerebrospinal fluid sample, naspharyngeal wash sample, sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumor sample, a tissue sample, a cell sample, a cell lysate sample, a virus culture sample, a forensic sample, an infection sample, a nosocomial infection sample, and any combinations thereof.

44. The method of claim 1, wherein said outer phase is surrounding said inner phase as a film.

45. The method of claim 3, wherein the at least one magnetically attractable particle comprises a surface-functionalized magnetically attractable particle.

46. A system for amplifying a nucleic acid molecule, said system comprising:
(a) at least one surface for receiving a first fluid droplet, said fluid droplet comprising an inner phase and an outer phase,
wherein the outer phase is immiscible with the inner phase, and the outer phase is surrounding the inner phase, wherein the inner phase comprises a sample comprising or suspected of comprising said nucleic acid molecule, and the inner phase is shielded from the environment by the outer phase, wherein said inner phase comprises surface functionalized magnetically attractable matter;
(b) at least one heater for heating a respective temperature zone on said at least one surface;
(c) means for controlling the position of said magnetically attractable matter relative to said surface so as to (1) purify said nucleic acid molecule; and (2) locate said magnetically attractable matter substantially onto said temperature zone; and
(d) means for amplifying said nucleic acid molecule.

47. The system of claim 46, wherein said means for controlling the position of said magnetically attractable matter relative to said surface comprise a magnetic or an electromagnetic field.

48. The system of claim 47, wherein said means for controlling the position of said magnetically attractable matter relative to said at least one surface further comprise means for altering said magnetic or electromagnetic field, for moving said at least one surface, or a combination thereof.

49. The system of claim 48, wherein said means for altering said magnetic field comprises means for altering the position of at least one magnet.

50. The system of claim 49 further comprises providing at least one wash droplet on said at least one surface.

51. The system of claim 46, wherein said heater comprises Platinum or Silicon.

52. The system of claim 46, further comprising providing at least a second heater for heating at a respective at least second temperature zone, wherein said temperature zones are located on the same surface.

53. The system of claim 52 further comprising providing at least a third heater for heating at a respective at least third temperature zone, wherein said temperature zones are located on the same surface.

54. The system of claim 53 further comprising providing at least a fourth heater for heating at a respective at least fourth temperature zone, wherein said temperature zones are located on the same surface.

55. The system of claim 53, further comprising means to independently control the temperature in each of said temperature zones.

56. The system of claim 54, further comprising means to independently control the temperature in each of said temperature zones.

57. The system of claim 46, wherein said system further comprises means to detect fluorescence.

58. The system of claim 46, wherein said amplification process comprises a reverse-transcriptase (RT), polymerase chain reaction (PCR), RT-PCR, a real-time quantitative PCR (qPCR), real-time quantitative RT-PCR (qRT-PCR), helicase dependent amplidication (tHDA), smart amplification process (SMAP), loop-mediated amplification (LAMP), rolling circle amplification (RCA), or recombinase polymerase amplification (RPA).

59. The system of claim 46, wherein said at least one surface comprises one surface.

60. A method for amplifying a nucleic acid molecule, said method comprising
(a) providing at least one surface for receiving a sample comprising or suspected of comprising said nucleic acid molecule, said at least one surface comprising a plurality of temperature zones at which temperature can be independently regulated, each temperature zone being located at a different location on said at least one surface;
(b) disposing said sample onto said at least one surface; and
(c) amplifying said nucleic acid molecule by moving said sample between said plurality of temperature zones, wherein said sample has a residency time at each temperature zone which is independently controlled.

61. The method of claim 60, wherein said residency time at least one of said plurality of temperature zones is independently controlled to vary between at least two sample movements onto the same temperature zone.

62. The method of claim 61, wherein said at least two sample movements comprise two consecutive sample movements, and wherein said residency time is independently controlled to vary between said two consecutive sample movements.

63. The method of claim 60, wherein said residency time at least one of said plurality of temperature zones is independently controlled to gradually increase or decrease between at least two sample movements onto the same temperature zone.

64. The method of claim 63, wherein said at least two sample movements comprise two consecutive sample movements, and wherein said residency time is independently controlled to vary between said two consecutive sample movements.

65. The method of claim 60, wherein at least one of said plurality of temperature zones said temperature is independently controlled to vary between at least two sample movements onto the same temperature zone.

66. The method of claim 63, wherein said at least two sample movements comprise two consecutive sample movements, and wherein said temperature is independently controlled to gradually increase or decrease between said two consecutive sample movements.

67. The method of claim 60, wherein at least one of said plurality of temperature zones said residency time and said temperature are both independently controlled to vary between at least two sample movements onto the same temperature zone.

68. The method of claim 67, wherein said at least two sample movements comprise two consecutive sample movements, and wherein said residency time and said temperature are independently controlled to vary between said two consecutive sample movements.

69. The method of claim 60, wherein at least one of said plurality of temperature zones said residency time and said temperature are both independently controlled to gradually increase or decrease between at least two sample movements onto the same temperature zone.

70. The method of claim 69, wherein said at least two sample movements comprise two consecutive sample movements, and wherein said residency time and said temperature are independently controlled to increase or decrease between said two consecutive sample movements.

71. The method of claim 60, further comprising providing means to detect fluorescence.

72. The method of claim 71, wherein the means to detect fluorescence are for detecting fluorescence when said sample is located onto at least one of said plurality of temperature zones.

73. The method of claim 60, wherein said amplifying comprises a reverse-transcriptase (RT), polymerase chain reaction (PCR), RT-PCR, a real-time quantitative PCR (qPCR), real-time quantitative RT-PCR (qRT-PCR), helicase dependent amplidication (tHDA), smart amplification process (SMAP), loop-mediated amplification (LAMP), rolling circle amplification (RCA), or recombinase polymerase amplification (RPA).

74. The method of claim 60, wherein said at least one surface comprises one surface.

75. The method of claim 60, wherein said sample comprises magnetic attractable matter and wherein moving said sample comprises controlling the position of said magnetic attractable matter relative to said at least one surface.

76. The method of claim 75, wherein the magnetically attractable matter is at least one magnetically attractable particle.

77. The method of claim 76, wherein the at least one magnetically attractable particle comprises diamagnetic particle, a ferromagnetic particle, a paramagnetic particle, a superparamagnetic particle, or any combinations thereof, and wherein said magnetically attractable particle is optionally bound to said nucleic acid molecule during said amplifying.

78. The method of claim 75, wherein controlling the position of said magnetically attractable matter relative to said at least one surface comprises exposing said magnetically attractable matter to a magnetic or an electromagnetic field.

79. The method of claim 78, wherein controlling the position of said magnetically attractable matter relative to said at least one surface further comprises moving the magnetically attractable matter by altering said magnetic or electromagnetic field, moving said at least one surface, or a combination thereof

80. The method of claim 79, wherein altering said magnetic field comprises altering the position of at least one magnet.

81. The method of claim 78, wherein controlling the position of said magnetically attractable matter further comprises moving said magnetically attractable matter by means of said magnetic or electro magnetic field.

82. The method of claim 63, wherein said at least two sample movements comprise two consecutive sample movements, and wherein said temperature is independently controlled to vary between said two consecutive sample movements.

83. The method of claim 60, wherein at least one of said plurality of temperature zones said temperature is independently controlled to gradually increase or decrease between at least two sample movements onto the same temperature zone.


SEQUENCE LISTING (11 sequences)

SEQ ID NO: 1 - protein, 9 aa, artificial
  sequence derived from FLAG peptide
DYKDDDDKG

SEQ ID NO: 2 - protein, 10 aa, artificial
  sequence derived from T7
ASMTGGQQMG

SEQ ID NO: 3 - protein, 11 aa, artificial
  sequence derived from HSV glycoprotein D
QPELAPEDPED

SEQ ID NO: 4 - protein, 11 aa, artificial
  sequence derived from VSV-G
YTDIEMNRLGK

SEQ ID NO: 5 - protein, 9 aa, artificial
  sequence derived from hemagglutinin
YPYDVPDYA

SEQ ID NO: 6 - protein, 10 aa, artificial
  sequence derived from c-myc
EQKLISEEDL

SEQ ID NO: 7 - DNA, 22 bp, artificial
  synthetic primer derived from pmaxGFP vector
atgaccaacaagatgaagagca

SEQ ID NO: 8 - DNA, 22 bp, artificial
  synthetic primer derived from pmaxGFP vector
gtaggtgccgaagtggtagaag

SEQ ID NO: 9 - DNA, 25 bp, artificial
  synthetic primer derived from pmaxGFP vector
aaggcgccctgaccttcagccccta

SEQ ID NO: 10 - DNA, 24 bp, artificial
  synthetic primer derived from hemagglutinin
caaacagattagtccttgcgactg

SEQ ID NO: 11 - DNA, 22 bp, artificial
  synthetic primer derived from hemagglutinin
cytgccatcctccctctataaa